(19) United States
(12) Patent Application Publication
(10) Pub. No.: US 2006/0257322 A1
(43) Pub. Date: Nov. 16, 2006

(54) Pharmaceutical agents that contain fluoroalkyl-containing metal complexes and epothilones

(76) Inventors: Ruediger Lawaczeck, Berlin (DE); Rolf Ruediger Press, Berlin (DE); Katja Schoen, Berlin (DE); Jens Hoffmann, Muehlenbeck (DE); Ulirch Klar, Berlin (DE); Bernd Misselwitz, Glienicke (DE); Johannes Platzek, Berlin (DE); Heiko Schirmer, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/355,251

(22) Filed: Feb. 16, 2006

Related U.S. Application Data
(60) Provisional application No. 60/657,694, filed on Mar. 3, 2005.

(30) Foreign Application Priority Data
Feb. 17, 2005 (DE) 102005008309.9

(51) Int. Cl. A61K 49/08; C07F 5/00

(52) U.S. Cl. 424/9.363; 424/9.364; 534/15; 536/17.1

(57) ABSTRACT

The invention relates to pharmaceutical agents that contain at least one fluoroalkyl-containing metal complex that has a critical micelle-formation concentration of <10⁻³ mol/l and a hydrodynamic micelle diameter (2Rh) of >1 nm, and at least one natural or synthetic epothilone or epothilone derivative, optionally with the additives that are commonly used in galenicals.

This application claims the benefit of the filing date of U.S. Provisional Application Ser. No. 60/657,694 filed Mar. 3, 2005, which is incorporated by reference herein

### DESCRIPTION

The invention relates to the subjects that are characterized in the claims: pharmaceutical agents that contain at least one fluoroalkyl-containing metal complex that has a critical micelle-formation concentration of <10⁻³ mo/l and a hydrodynamic diameter (2Rh) of >1 nm, and at least one natural or synthetic epothilone or epothilone derivative, and their use for diagnosis, therapy and monitoring of the course of therapy of tumors, in particular by intravenous administration.

Tumor therapy faces a number of problems that mostly arise from the fact that the selected therapy modality is not directed solely at the tumor and its metastases but generally also affects the surrounding normal tissue. The unavoidable damage of the normal tissue, which surrounds the tumor, or of the entire organism, in many cases makes it impossible to administer substance or radiation in doses that are sufficient for tumor treatment. By physical measures, the radiation therapy can be focused on the tumor, but the surrounding tissue cannot be completely kept out of the radiation field. Similar arguments hold true for the local administration of cytostatic agents such as epothilones and epothilone derivatives. Comparable complications also follow in chemotherapy by inadequate tumor specificity of the compounds.

In the past, a number of transport mechanisms (e.g., vehicles) were examined that are to introduce chemotherapeutic agents purposefully into the tumor. In Patent Application WO 97/30969, for example, pharmaceutical agents are described that contain a fluoroalkyl-containing compound. By the thixotropic nature of these compounds, the formation of a locally limited, temporary vascular occlusion (embolization) results after local, intravascular administration. In the application, it is further disclosed that the compositions can contain additives for interventional radiology or for tumor therapy, e.g., chemotherapy agents (in particular cytostatic agents, e.g., 5-fluorouracil, cisplatin, doxorubicin, or mitomycin C). By the combination (embolizing agent and chemotherapy agent), it is achieved that the therapeutic agent is slowly released from the embolus over an extended period (chemoembolization).

This method has the drawback, however, that the tumor can only be addressed locally, since the compositions immediately produce an occlusion of the vessel (embolus) at the site of the administration. The cytostatic agent that is contained in the composition then also diffuses only locally at the site of the occlusion into the adjacent tissue. The target-specific, local administration requires the insertion of a catheter selectively into the tumor-supplying blood vessel. Because of the high viscosity of the compositions, the latter must be administered under high pressure. This is therefore a greatly invasive treatment method that is very uncomfortable for the patients and is associated with the increased risk of side effects.

The method has the drawback, moreover, that the therapeutic effect can occur only locally—close to the embolus—and thus, on the one hand, tumor metastases are not detected and, on the other hand, the danger exists that the embolus is not formed specifically on the tumor and the therapy therefore only inadequately reaches the tumor.

In addition, the method requires the presence of a sufficiently large vessel that supplies the tumor with blood to make a catheterization possible at all. This greatly limits the selection of the types of tumors that can be treated with this method; this process is basically therefore only used for treating liver tumors.

The object of the invention is thus to make available pharmaceutical agents that on the one hand exhibit high effectiveness and specificity relative to tumors, address all regions of the body and therefore also treat satellite metastases, and at the same time are as gentle as possible to the patients, especially with respect to the administration of the pharmaceutical agents. This object is achieved by the invention.

It has been found, surprisingly enough, that pharmaceutical agents that contain at least one fluoroalkyl-containing metal complex, which has a critical micelle-formation concentration of <10⁻³ mol/l and a hydrodynamic diameter (2Rh) of >1 nm, and at least one natural or synthetic epothilone or epothilone derivative, are very well suited for diagnosis, therapy and monitoring of the course of therapy of tumors. It has been found that the therapeutic agents according to the invention show a surprisingly high effectiveness for combating tumors (see Tumor Therapy Tests 1 to 16) and at the same time show decisive advantages in the administration and dispersion: the agents are solutions of comparatively low viscosity (Example 1) that can be dispersed in the entire body because of the systemic (intravenous) administration (Example 2) and thus also can address satellite metastases. This intravenous administration (non-invasive) has clear advantages for patients with respect to discomfort in comparison to administration via catheter (as is necessary for the agents that are disclosed in WO 97/30969).

It was found, moreover, that fluoroalkyl compounds, which are described in, for example, Patent Applications WO 02/14309 and WO 97/30969, have the capacity to mediate the solution of hydrophobic substances, e.g., epothilones, in aqueous systems and accordingly to transport their pharmacokinetic and pharmacodynamic properties, i.e., without in this case essentially altering their distribution behavior. The structures that develop have a smaller hydrodynamic diameter than, for example, liposomes and are trapped only in a small portion of MPS in the liver and spleen. The therapeutic agent (e.g., natural or synthetic epothilone or epothilone derivatives) is transported with the aid of the fluoroalkyl-containing compound to the target site (tumor) and has a prolonged dwell time there. The micellar encapsulation results in an elevated stability of epothilones in an aqueous medium. The local therapy effect (tumor monitoring, partial or complete tumor emission) can be tracked without renewed administration of a diagnostic agent with the corresponding radiological process, since its long dwell time extends over a chemotherapy cycle. The fluoroalkyl-containing metal complex thus fulfills several functions: contrast media in imaging for the visualization of tumors, carrier for the epothilones to the site of action (tumor) and contrast media for therapy monitoring.

In imaging diagnosis, only a few substances that show a direct tumor concentration are available. In animal-experimental studies, it was possible to show that the above-mentioned fluoroalkyl-containing metal complexes preferably accumulate in tumors and dwell for a long time there. As paramagnetic compounds, the pharmaceutical agents according to the invention are therefore also very well suited for imaging diagnosis of tumors (Example 2, FIG. 1).

It has now turned out, surprisingly enough, that specifically these substances with prolonged dwell times in the tumor can also be readily charged with therapeutic agents, especially natural or synthetic epothilones or epothilone derivatives and, on the one hand, are suitable as vehicles for tumor concentration, and, on the other hand, are extremely well suited for intravenous administration and addressing of metastases. They can thus focus the therapeutic action on the tumors and the metastases present in the body, can be administered intravenously in a simple and uncomplicated manner, and by the dispersion in the body, there is no danger of the tumor not being reached.

The therapeutic effectiveness of the agents according to the invention can be enhanced by x rays or other rays that cause an accumulation or release of the active substance in the tumor, or their action can be increased. (Solberg, T. D.; Iwamoto, K. S.; Norman, A. Calculation of Radiation Dose Enhancement Factors for Dose Enhancement Therapy of Brain Tumors. Phys Med Biol. (1992), 37: 439-443).

The pharmaceutical agents according to the invention that consist of at least one fluoroalkyl-containing compound and at least one natural or synthetic epothilone or epothilone derivative usually have a hydrodynamic diameter of ≧1 nm. In particular, those fluoroalkyl-containing metal complexes according to the invention whose 2 Rh ≧2 nm, preferably ≧3 nm, are suitable. The hydrodynamic diameter is determined by dynamic photocorrelation spectroscopy. In the substances according to the invention, the critical micelle-formation concentration (CMC) is usually <10⁻³ mol/l, in especially suitable cases <10⁻⁴ mol/l, and in quite especially suitable cases <10⁻⁵ mol/l. The determination of the CMC is described in H.-D. Dörfler “Grenzflächen und Kolloidchemie [Interface and Colloid Chemistry]”; Weinheim, N.Y., Basel, Cambridge, Tokyo; VSH 1994.

As epothilone or epothilone derivates, the compounds that are mentioned in, for example, DE 19907588, WO 98/25929, WO 99/58534, WO 99/2514, WO 99/67252, WO 99/67253, WO 99/7692, EP 99/4915, WO 00/485, WO 00/1333, WO 00/66589, WO 00/49019, WO 00/49020, WO 00/49021, WO 00/71521, WO 00/37473, WO 00/57874, WO 01/92255, WO 01/81342, WO 01/73103, WO 01/64650, WO 01/70716, U.S. Pat. No. 6,204,388, U.S. Pat. No. 6,387,927, U.S. Pat. No. 6,380,394, U.S. Ser. No. 02/52,028, U.S. Ser. No. 02/58,286, U.S. Ser. No. 02/62030, WO 02/32844, WO 02/30356, WO 02/32844, WO 02/14323, and WO 02/8440 can be used.

Epothilones or epothilone derivatives that can preferably be used are described in Claim 50. Epothilones and epothilone derivatives that can especially preferably be used are described in Table 2.

In DE 100 41470A1, DE 199 21 086A1 and DE 199 23 001A1, epothilone and epothilone derivatives are proposed for combination therapy with additional substance classes that can be used in tumor therapy, such as, for example, platinum complexes, e.g., cisplatin. Here, a combination therapy means, however, the administration, separated in time, of two therapeutically active principles; i.e., two monotherapies are performed together: in this case, the treatment, on the one hand, with a cytostatic agent (epothilone or epothilone derivative), and, on the other hand, with the cytotoxic substance cisplatin, which binds to DNA and thus primarily kills quick-growing cells.

It was not proposed in the documents, however, to combine the fluoroalkyl-containing metal complexes of this invention with epothilones in a single preparation as a formulation for epothilones. The fluoroalkyl-containing metal complexes are now distinguished by especially good compatibility and themselves have no anti-tumor action.

In an embodiment of this invention, the compounds of general formula I according to claims 7 to 10 are used as preferred compounds. In this case, these are known compounds that are described in WO 97/26017. Also, their production can be found in this WO publication. Surprisingly enough, it has been shown that these compounds also together with epothilones or epothilone derivatives are very well suited for diagnosis, therapy and monitoring of the course of therapy of tumors. Metal complexes 1-4, 6 and 11-13 (cf. also Table 1) are used as quite especially preferred compounds.

In another embodiment of this invention, those compounds of general formula Ia according to claims 11 to 20 are used as preferred compounds. These compounds are known and are described in WO 99/01161. Their use according to this invention has not yet been described. Of these compounds, metal complex 14 (cf. Table 1) is quite especially preferably used.

In other preferred embodiments of the invention, the compounds of general formula Ib according to claims 21 to 28, of general formula Ic according to claims 29 to 35, and of general formula Id according to claims 36 to 43 can be used. These compounds and their production are described in DE 100 40 380 (WO 02/13874).

In another preferred embodiment of the invention, galenical formulations that contain paramagnetic and diamagnetic fluoroalkyl-containing substances can be used. The paramagnetic and diamagnetic substances are preferably present in a dissolved state in an aqueous solvent.

As paramagnetic, fluoroalkyl-containing compounds, all above-mentioned metal complexes of general formulas I, Ia, Ib, Ic and/or Id can be used in the formulations according to the invention.

The diamagnetic, perfluoroalkyl-containing substances are those according to claims 45 to 49; they are also described—exactly like the above-mentioned galenical formulations—in Patent Application DE 100 40 380 (WO 02/13874).

In summary, it is noted that gadolinium complexes 1-16 that are cited in Table 1 meet the criteria according to the invention as quite especially preferred compounds.

Both the paramagnetic compounds of general formulas I, Ia, Ib, Ic and Id according to the invention and the formulations that consist of paramagnetic and diamagnetic fluoroalkyl-containing substances according to the invention are extremely well suited together with epothilones or epothilone derivatives for diagnosis, therapy and the monitoring of the course of therapy of tumors.

The term “metal ion equivalent” as used in the claims is a common term that is known to one skilled in the art in the field of complex chemistry. A metal ion equivalent is an equivalent of metal ions that can bind to, e.g., a carboxylate group instead of hydrogen. For example, a Gd³⁺ can bind to 3 carboxylate groups, i.e., ⅓ Gd³⁺ corresponds to metal ion equivalent R¹ in formula (III) (see Claim 7) if the metal is gadolinium.

Preferred fluorine complex: epothilone molecule ratios are 100:1 to 1:1, especially preferred are the ratios of 20:1 to 5:1.

In an especially preferred embodiment, the pharmaceutical agents according to the invention are administered intravenously.

The invention therefore relates to pharmaceutical agents according to the invention for intravenous administration. In a preferred embodiment, the latter are used in the therapy of tumors. In another preferred embodiment, the latter are used in the graphic visualization of tumors, especially in the monitoring of the course of therapy in the treatment of tumors.

In addition, the invention relates to the use of pharmaceutical agents according to the invention for the production of a medication or diagnostic agent for intravenous administration. In a preferred embodiment, the latter are used in the therapy of tumors. In another preferred embodiment, the latter are used in the graphic visualization of tumors, especially in the monitoring of the course of therapy in the treatment of tumors.

In addition, the agents according to the invention can be used in combination with an additional radiation therapy for the therapy of tumors.

In addition, the invention relates to a process for treating tumors, characterized in that the agents according to the invention are administered intravenously to the patient in a pharmaceutically effective amount.

In addition, the invention relates to a process for imaging visualization of tumors, characterized in that the agents according to the invention are administered intravenously to the patient and the metal complex that is contained in the agent is detected in the body by means of a suitable system, whereby a local increase of the signal shows the presence of the tumor. The detection can take place by, e.g., MRI or CT based on the metal ion that is contained in the metal complex. In a preferred embodiment, the imaging visualization of tumors can be used for the monitoring of the course of therapy of tumors. In this case, the process for imaging visualization of tumors is used at least two times at the beginning of the tumor treatment and/or during the tumor treatment and/or after the end of the tumor treatment, and the result of the later detection is compared to the earlier detection(s). The course of the therapy thus can be monitored.

### FIGURES

FIG. 1: FIG. 1 shows an MR cross-sectional image (axial) for 24 hours (a), 192 hours (b), as well as 336 hours (c) after i.v. administration of 200 μmol of .Gd/kg of body weight of the formulation of Example 1, F1-E (T:Tumor; N:Tumor necrosis).

The examples that are indicated below are used to explain the subject of the invention.

### EXAMPLES

The table presented below provides a list of the metal complexes that are preferably used for a production of a formulation.

TABLE 1
Gd-Metal Complexes Preferably Used According to the Invention for the
Production of Epothilone Formulations

| Metal | |
|---|---|
| Complex | Origin/Bibliographic Reference, Name |
| 1 | WO 97/26017, Example 33f |
| | Gadolinium Complex of 10-[1-Methyl-2-oxo-3-aza-5-oxo-{4-perfluorooctyl- |
| | sulfonyl-piperazin-1-yl}-pentyl]-1,4,7-tris(carboxymethyl)-1,4,7,10- |
| | tetraazacyclododecane |
| 2 | WO 97/26017, Example 2c |
| | Gadolinium Complex of 10-[2-Hydroxy-4-aza-5-oxo-7-oxa-10,10,11,11, |
| | 12,12,13,13,14,14,15,15,16,16,17,17-heptadecafluoroheptadecyl]-1,4,7- |
| | tris(carboxymethyl)-1,4,7,10-tetraazacyclododecane |
| 3 | WO 97/26017, Example 34b |
| | Gadolinium Complex of 10-[2-Hydroxy-4-aza-5,9-dioxo-9-{4- |
| | perfluorooctyl)-piperazin-1-yl}-nonyl]-1,4,7-tris(carboxymethyl)-1,4,7,10- |
| | tetraazacyclododecane |
| 4 | WO 97/26017, Example 1c |
| | Gadolinium Complex of 10-[2-Hydroxy-4-aza-5-oxo-7-aza-7-(perfluorooctyl- |
| | sulfonyl)-nonyl]-1,4,7-tris(carboxymethyl)-1,4,7,10-tetraazacyclododecane |
| 5 | WO 02/13874, Example 2c |
| | 1,4,7-Tris(carboxylatomethyl)-10-(3-aza-4-oxo-hexan-5-ylic)-acid-N-2,3- |
| | dihydroxypropyl)-N-(1H,1H,2H,2H,4H,4H,5H,5H-3-oxa)- |
| | perfluorotridecyl)-amide]-1,4,7,10-tetraazacyclododecane, Gadolinium |
| | Complex |
| 6 | WO 97/26017, Example 3c |
| | Gadolinium Complex of 10-[2-Hydroxy-4-oxa-1H,1H,2H,3H,3H,5H,5H, |
| | 6H,6H-perfluorotetradecyl]-1,4,7-tris(carboxymethyl)-1,4,7,10- |
| | tetraazacyclododecane |
| 7 | WO 02/13874, Example 5e |
| | 1,4,7-Tris(carboxylatomethyl)-10-{(3-aza-4-oxo-hexan-5-ylic)acid-[N- |
| | (3,6,9,12,15-pentaoxa)-hexadecyl)-N-(1H,1H,2H,2H,4H,4H,5H,5H-3- |
| | oxa)-perfluorotridecyl]-amide}-1,4,7,10-tetraazacyclododecane, Gadolinium |
| | Complex |
| 8 | WO 02/13874, Example 3c |
| | 1,4,7-Tris(carboxylatomethyl)-10-{(3-aza-4-oxo-hexan-5-ylic)-acid-N-(5- |
| | hydroxy-3-oxa-pentyl)-N-(1H,1H,2H,2H,4H,4H,5H,5H-3-oxa)- |
| | perfluorotridecyl]-amide}-1,4,7,10-tetraazacyclododecane, Gadolinium |
| | Complex |
| 9 | WO 02/13874, Example 6b |
| | 1,4,7-Tris(carboxylatomethyl)-10-{(3-aza-4-oxo-hexan-5-ylic)-acid-[N- |
| | 3,6,9,16-tetraoxa-13-aza-14-oxo-C₁₉-C₂₆-hepta-decafluoro)hexacosyl]- |
| | amide}-1,4,7,10-tetraazacyclododecane, Gadolinium Complex |
| 10 | WO 02/13874, Example 1c |
| | 1,4,7-Tris(carboxylatomethyl)-10-[(3-aza-4-oxo-hexan-5-ylic]-acid-N-(2- |
| | methoxyethyl)-N-(1H,1H,2H,2H,4H,4H,5H,5H-3-oxa)- |
| | perfluorotridecyl]-amide}-1,4,7,10-tetraazacyclododecane, Gadolinium |
| | Complex |
| 11 | WO 97/26017, Example 32c |
| | Gadolinium Complex of 10-[2-Hydroxy-4-aza-5-oxo-7-oxa- |
| | 10,10,11,11,12,12,13,13,14,14,15,15,16,16,17,17,18,18,19,19- |
| | henicosafluoro-nonadecyl]-1,4,7-tris(carboxymethyl)-1,4,7,10- |
| | tetraazacyclododecane |
| 12 | WO 97/26017, Example 38d |
| | Gadolinium Complex of 10-[2-Hydroxy-4-aza-5-oxo-11-aza-11-(perfluoro- |
| | octylsulfonyl)-tridecyl]-1-4-7-tris(carboxymethyl) 1,4,7,10- |
| | tetraazacyclododecane |
| 13 | WO 97/26017, Example 35d |
| | Gadolinium Complex of 10-[2-Hydroxy-4-aza-5-oxo-7-aza-7-(perfluorooctyl- |
| | sulfonyl)-8-phenyl-octyl]-1-4-7-tris(carboxymethyl)-1,4,7,10-tetraazacyclododecane |
| 14 | WO 99/01161, Example 1g |
| | 1,4,7-Tris{1,4,7-tris(N-(carboxylatomethyl)-10-[N-1-methyl-3,6-diaza-2,5,8- |
| | trioxooctane-1,8-diyl]-1,4,7,10-tetraazacyclododecane, Gd complex}-10-N- |
| | 2H,2H,4H,4H,5H,5H-3-oxa-perfluoro-tridecanoyl]-1,4,7,10-tetraazacyclododecane, |
| | Gd Complex |
| 15 | WO 02/13874, Example 21f = Example 1f in WO 02/14309 |
| | 6-N-[1,4,7-Tris(carboxylatomethyl)-1,4,7,10-tetraazacyclododecane-10-N- |
| | (pentanoyl-3-aza-4-oxo-5-methyl-5-yl)]-2-N-[1-O-α-D-carbonylmethyl- |
| | mannopyranose]-L-lysine-[1-(4-perfluorooctylsulfonyl)-piperazine]-amide, |
| | Gd Complex |
| 16 | WO 02/13874, Example 54b, |
| | 2,6-N,N′-Bis[1,4,7-tris(carboxylatomethyl)-1,4,7,10-tetraazacyclododecane- |
| | 10-(pentanoyl-3-aza-4-oxo-5-methyl-5-yl)]-lysine-[1-(4- |
| | perfluorooctylsulfonyl-piperazine]-amide, Gd Complex |
| 17 | Dysprosium complex of 10-[1-methyl-2-oxo-3-aza-5-oxo-{4-perfluorooctylsulfonyl- |
| | piperazin-1-yl}-pentyl]-1,4,7-tris(carboxymethyl)-1,4,7,10- |
| | tetraazacyclododecane |
| | Three molar equivalents of oxalic acid are added to an aqueous solution of |
| | the gadolinium complex of 10-[1-methyl-2-oxo-3-aza-5-oxo-{4- |
| | perfluorooctylsulfonyl-piperazin-1-yl}-pentyl]-1,4,7-tris(carboxymethyl)- |
| | 1,4,7,10-tetraazacyclododecane (WO 97/26017, Example 33f), and it is |
| | heated for 6 hours to 90° C. Precipitating gadolinium oxalate is filtered out, |
| | and the free ligand remaining in the filtrate is complexed with dysprosium |
| | oxide. The crude product is purified by chromatography on RP-18 silica gel |
| | and then freeze-dried. |
| 18 | Ytterbium complex of 10-[1-methyl-2-oxo-3-aza-5-oxo-{4-perfluorooctylsulfonyl- |
| | piperazin-1-yl}-pentyl]-1,4,7-tris(carboxymethyl-1,4,7,10- |
| | tetraazacyclododecane |
| | Three molar equivalents of oxalic acid are added to an aqueous solution of |
| | the gadolinium complex of 10-[1-methyl-2-oxo-3-aza-5-oxo-{4- |
| | perfluorooctylsulfonyl-piperazin-1-yl}-pentyl]-1,4,7-tris(carboxymethyl)- |
| | 1,4,7,10-tetraazacyclododecane (WO 97/26017, Example 33f), and it is |
| | heated for 6 hours to 90° C. Precipitating gadolinium oxalate is filtered out, |
| | and the free ligand remaining in the filtrate is complexed with ytterbium |
| | oxide. The crude product is purified by chromatography on RP-18 silica gel |
| | and then freeze-dried. |
| 19 | Yttrium complex of 10-[1-methyl-2-oxo-3-aza-5-oxo-{4-perfluorooctylsulfonyl- |
| | piperazin-1-yl}-pentyl]-1,4,7-tris(carboxymethyl)-1,4,7,10- |
| | tetraazacyclododecane |
| | Three molar equivalents of oxalic acid are added to an aqueous solution of |
| | the gadolinium complex of 10-[1-methyl-2-oxo-3-aza-5-oxo-{4- |
| | perfluorooctylsulfonyl-piperazin-1-yl}-pentyl]-1,4,7-tris(carboxymethyl)- |
| | 1,4,7,10-tetraazacyclododecane (WO 97/26017, Example 33f), and it is |
| | heated for 6 hours to 90° C. Precipitating gadolinium oxalate is filtered out, |
| | and the free ligand remaining in the filtrate is complexed with yttrium oxide. |
| | The crude product is purified by chromatography on RP-18 silica gel and then |
| | freeze-dried. |

The table that is indicated below provides a list of epothilones A-N that are used in these examples:

TABLE 2
Epothilones That are Quite Especially Preferably Used According to the
Invention

| | Source | Example | Compound Name |
|---|---|---|---|
| A | WO 00/49021 | 1 | (4S,7R,8S,9S,13(Z),16S(Z)-4,8-Dihydroxy-16-(1-fluoro-2- |
| | | | (2-methyl-4-thiazolyl)ethenyl)-1-oxa-5,5,7,9,13- |
| | | | pentamethyl-cyclohexadec-13-ene-2,6-dione |
| B | WO 00/49021 | 5 | (4S,7R,8S,9S,13(Z),16S(Z)-4,8-Dihydroxy-16-(1-chloro-2- |
| | | | (2-methyl-4-thiazolyl)ethenyl)-1-oxa-5,5,7,9,13- |
| | | | pentamethyl-cyclohexadec-13-ene-2,6-dione |
| C | WO 00/49021 | 7(A) | (1S,3S(Z),7S,10R,11S,12S,16R)-7,11-Dihydroxy-3-(1- |
| | | | chloro-2-(2-methyl-4-thiazolyl)ethenyl)-8,8,10,12,16- |
| | | | pentamethyl-4,17-dioxabicyclo[14.1.0]heptadecane-5,9- |
| | | | dione |
| D | WO 00/49021 | 7(B) | (1S,3S(Z),7S,10R,11S,12S,16S)-7,11-Dihydroxy-3-(1- |
| | | | chloro-2-(2-methyl-4-thiazolyl)ethenyl)-8,8,10,12,16- |
| | | | pentamethyl-4,17-dioxabicyclo[14.1.0]heptadecane-5,9- |
| | | | dione |
| E | WO 00/49021 | 13 | 4S,7R,8S,9S,13(Z),16S(Z)-4,8-Dihydroxy-9,13-dimethyl- |
| | | | 7-ethyl-16-(1-fluoro-2-(2-methyl-4-thiazolyl)ethenyl-1- |
| | | | oxa-5,5-(1,3-trimethylene)-cyclohexadec-13-ene-2,6-dione |
| F | WO 00/66589 | 31(A) | (1S,3S,7S,10R,11S,12S,16R)-7,11-Dihydroxy-3-(2- |
| | | | methylbenzothiazol-5-yl)-10-(prop-2-en-1-yl)-8,8,12,16- |
| | | | tetramethyl-4,17-dioxabicyclo[14.1.0]heptadecane-5,9- |
| | | | dione |
| G | WO 00/49021 | 17 | 4S,7R,8S,9S,13(Z),16S(Z)-4,8-Dihydroxy-16-(1-fluoro-2- |
| | | | (2-pyridyl)ethenyl)-7-ethyl-1-oxa-5,5,9,13-tetramethyl- |
| | | | cyclohexadec-13-ene-2,6-dione |
| H | WO 00/66589 | 30 | (4S,7R,8S,9S,13(E),16S)-4,8-Dihydroxy-16-(2- |
| | | | methylbenzothiazol-5-yl)-1-oxa-5,5,9,13-tetramethyl-7- |
| | | | (prop-2-en-1-yl)-cyclohexadec-13-ene-2,6-dione |
| I | WO 00/66589 | 1 | (4S,7R,8S,9S,13(Z),16S(E))-4,8-Dihydroxy-16-(1-methyl- |
| | | | 2-(2-pyridyl)ethenyl)-1-oxa-5,5,9,13-tetramethyl-7-(but-3- |
| | | | in-1-yl)-cyclohexadec-13-ene-2,6-dione |
| J | WO 00/66589 | 2 | (4S,7R,8S,9S,13(Z),16S(E))-4,8-Dihydroxy-16-(1-methyl- |
| | | | 2-(2-pyridyl)ethenyl)-1-oxa-5,5,9,13-tetramethyl-7-(but-3- |
| | | | en-1-yl)-cyclohexadec-13-ene-2,6-dione |
| K | WO 00/66589 | 9 | (4S,7R,8S,9S,13(Z),16S(E))-4,8-Dihydroxy-16-(1-methyl- |
| | | | 2-(2-methylthiazol-4-yl)ethenyl)-1-oxa-5,5,9,13- |
| | | | tetramethyl-7-(prop-2-en-1-yl)-cyclohexadec-13-ene-2,6- |
| | | | dione |
| L | WO 00/66589 | 23 | (4S,7R,8S,9S,13(Z),16S(Z))-4,8-Dihydroxy-16-(1-fluoro- |
| | | | 2-(2-methylthiazol-4-yl)ethenyl)-7-(prop-2-in-1-yl)-1-oxa- |
| | | | 5,5,9,13-tetramethyl-cyclohexadec-13-ene-2,6-dione |
| M | WO 00/66589 | 25(A) | (1S,3S(Z),7S,10R,11S,12S,16R)-7,11-Dihydroxy-3-(1- |
| | | | fluoro-2-(2-methyl-4-thiazolyl)ethenyl)-10-(prop-2-in-1- |
| | | | yl)-8,8,12,16-tetramethyl-4,17- |
| | | | dioxabicyclo[14.1.0]heptadecane-5,9-dione |
| N | WO 00/66589 | 27 | (4S,7R,8S,9S,13(Z),16S(Z))-4,8-Dihydroxy-16-(1-fluoro- |
| | | | 2-(2-methyloxazol-4-yl)ethenyl)-7-(prop-2-in-1-yl)-1-oxa- |
| | | | 5,5,9,13-tetramethyl-cyclohexadec-13-ene-2,6-dione |

### Example 1

### General Instructions for the Production of Pharmaceutical Agents

x mmol of epothilone (Table 2) is dissolved in 50 μl of ethanol (40° C.) and converted into y ml of a gadolinium complex solution (complex no.) from z mmol of Gd/I of the compound of Table 1.

On the site of instillation, a white fog forms spontaneously, and said fog disappears after a brief shaking. A clear solution is present.

TABLE 3
Formulations of Epothilones with Fluorine Chain-Containing Gd
Complexes that are Quite Especially Preferably Used
According to the Invention

| | | x mmol of Epothilone in y |
|---|---|---|
| | | ml of Gd Complex (z |
| Designation of the | Epothilone | mmol/l) |
| Formulation Under Study | Gd Complex No. | (Remarks) |
| F1-A | A | x: 0.0176 |
| | 12 | y: 8 |
| | | z: 20 |
| F1-B | B | x: 0.0176 |
| | 15 | y: 8 |
| | | z: 20 |
| F1-C | C | x: 0.0176 |
| | 15 | y: 8 |
| | | z: 20 |
| F1-D | D | x: 0.0176 |
| | 7 | y: 8 |
| | | z: 20 |
| F1-E | E | x: 0.0176 |
| | 15 | y: 8 |
| | | z: 20 |
| F1-F | F | x: 0.0176 |
| | 15 | y: 8 |
| | | z: 20 |
| F1-G | G | x: 0.0176 |
| | 4 | y: 8 |
| | | z: 20 |
| F1-H | H | x: 0.0176 |
| | 15 | y: 8 |
| | | z: 20 |
| F1-I | I | x: 0.0176 |
| | 3 | y: 8 |
| | | z: 20 |
| F1-K | K | x: 0.0176 |
| | 5 | y: 8 |
| | | z: 20 |
| F1-L | L | x: 0.0176 |
| | 4 | y: 8 |
| | | z: 20 |
| F1-M | M | x: 0.0176 |
| | 1 | y: 8 |
| | | z: 20 |
| F1-N | N | x: 0.0176 |
| | 5 | y: 8 |
| | | z: 20 |

### Example 2

### Tumor Visualization with Use of the Formulation of Example 1, F1-E

Tumor Inoculation: In nu/nu hairless mice, a human non-small-cell lung tumor was induced subcutaneously in the area of the right femur muscle by inoculation of A549 cells (4×10⁶ cells/30 μl of full medium). About 4 weeks after the tumor induction, the slow-growing tumor reached rice grain size. At this time, imaging was begun.

MR Imaging: (Siemens 1.5 T. SE sequence, TR/TE 400/15 ms, α 90°, axial, 7 layers). Images of tumors before (baseline) and 5, 10, 20 minutes, and 24, 96, 192 and 336 hours after intravenous (i.v.) administration of 200 μmol of Gd/kg of body weight of the formulation of Example 1, F1-E. Typical cross-sectional images are shown in FIG. 1. The cross-sectional images of FIG. 1 show the strong concentration (homogeneous enhancement) of the formulation in the tumor at time 24 hours p.i., as well as its slow abatement subsequently with simultaneous increase of signal intensity in tumor necrosis.

### Example 3

### Formulations of the Pharmaceutical Agents with Increased Epothilone Content

x mmol of epothilone (Tab. 2) is dissolved in 50 μl of ethanol (40° C.) and converted into y ml of a gadolinium complex solution that consists of z mmol of Gd/l of the compound of Table 1 (Complex No.). After being shaken for a short length of time, a clear homogeneous solution is produced.

TABLE 4
Formulations of Epothilones with Fluorine-Chain-Containing Gd
Complexes That are Quite Especially Preferably Used
According to the Invention (Increased EPO Portion)

| | | x mmol of Epothilone in y |
|---|---|---|
| Designation of the | Epothilone | ml of Gd Complex (z |
| Formulation Under Study | Gd Complex No. | mmol/l) (Remarks) |
| F2-A | A | x: 0.0235 |
| | 12 | y: 8 |
| | | z: 20 |
| F2-B | B | x: 0.0235 |
| | 15 | y: 8 |
| | | z: 20 |
| F2-C | C | x: 0.0235 |
| | 15 | y: 8 |
| | | z: 20 |
| F2-D | D | x: 0.0235 |
| | 7 | y: 8 |
| | | z: 20 |
| F2-E | E | x: 0.0235 |
| | 15 | y: 8 |
| | | z: 20 |
| F2-F | F | x: 0.0235 |
| | 15 | y: 8 |
| | | z: 20 |
| F2-G | G | x: 0.0235 |
| | 4 | y: 8 |
| | | z: 20 |
| F2-H | H | x: 0.0235 |
| | 15 | y: 8 |
| | | z: 20 |
| F2-I | I | x: 0.0235 |
| | 3 | y: 8 |
| | | z: 20 |
| F2-K | K | x: 0.0235 |
| | 5 | y: 8 |
| | | z: 20 |
| F2-L | L | x: 0.0235 |
| | 4 | y: 8 |
| | | z: 20 |
| F2-M | M | x: 0.0235 |
| | 1 | y: 8 |
| | | z: 20 |
| F2-N | N | x: 0.0235 |
| | 5 | y: 8 |
| | | z: 20 |

### Example 4

### Tumor Therapy Examples with Use of the Formulations of Examples 1 and 3

A549 lung tumors were induced in hairless mice (4×10⁶ cells/30 μl of full medium). Animal weights and tumor surface areas/volumes were determined, and in this case, the tumor surface area or the tumor volume (based on an ellipsoid of revolution) was calculated. Length and width of the tumor were measured with vernier calipers. After reaching the size of a pea, the treatment was begun.

### Description of the Formulations and Dosages Used By Way of Example

(Explanations regarding the information in Table 4):

Test Solutions:

(a) 20 mmol of Gd/l of the Gd complex compound as mentioned in Example 1 or 3 (as a comparison solution=Blank reading)
(b) 1.5 mmol/l of the epothilone as mentioned in Example 1 or 3, in aqueous hydroxypropyl-β-cyclodextrin solution (prior art) (For the production of formulations in the prior art, see WO 99/07692 and WO 00/4902 1)
(c) Formulation of Example 1 or 3 (formulation according to the invention)
   Dosages:
(a) 100 μmol of Gd/kg of body weight
(b) 11 μmol/kg or 18 μmol/kg of body weight
(c) 100 μmol of Gd/kg+11 (or 18) μmol of epothilone/kg of body weight (corresponds to the ratio in the formulation of Example 1 or 3).

The animal weights and tumor sizes were measured over a period of 20 days after intravenous administration.

Table 4 that is indicated below sets forth the dosages of tumor-therapy tests 1-16 that are used:

TABLE 4

| | (b) | (c) | |
|---|---|---|---|
| (a) | Prior Art | Formulation | Tumor- |
| Gd Complex as | Epothilone | According to the | Therapy |
| Comparison | Formulation | Invention | Test |
| Dosage | Dosage | Dosage | No. |
| 20 mmol of Gd/l of | 1.5 mmol of | F1-A | 1 |
| the complex | epothilone | 100 μmol of Gd/kg | |
| | | of body weight | |
| 100 μmol of Gd/kg | 11 μmol/kg of body | 11 μmol of EPO/kg | |
| of body weight | weight | of body weight | |
| 20 mmol of Gd/l of | 1.5 mmol of | F2-B | 2 |
| the Complex | epothilone | 100 μmol of Gd/kg | |
| | | of body weight | |
| 100 μmol of Gd/kg | 18 μmol/kg of body | 18 μmol of EPO/kg | |
| of body weight | weight | of body weight | |
| 20 mmol of Gd/l of | 1.5 mmol of | F1-C | 3 |
| the Complex | epothilone | 100 μmol of Gd/kg | |
| | | of body weight | |
| 100 μmol of Gd/kg | 11 μmol/kg of body | 11 μmol of EPO/kg | |
| of body weight | weight | of body weight | |
| 20 mmol of Gd/l of | 1.5 mmol of | F2-C | 4 |
| the complex | epothilone | 100 μmol of Gd/kg | |
| | | of body weight | |
| 100 μmol of Gd/kg | 18 μmol/kg of body | 18 μmol of EPO/kg | |
| of body weight | weight | of body weight | |
| 20 mmol of Gd/l of | 1.5 mmol of | F1-E | 5 |
| the complex | epothilone | 100 μmol of Gd/kg | |
| | | of body weight | |
| 100 μmol of Gd/kg | 11 μmol/kg of body | 11 μmol of EPO/kg | |
| of body weight | weight | of body weight | |
| 20 mmol of Gd/l of | 1.5 mmol of | F2-E | 6 |
| the complex | epothilone | 100 μmol of Gd/kg | |
| | | of body weight | |
| 100 μmol of Gd/kg | 18 μmol/kg of body | 18 μmol of EPO/kg | |
| of body weight | weight | of body weight | |
| 20 mmol of Gd/l of | 1.5 mmol of | F1-F | 7 |
| the complex | epothilone | 100 μmol of Gd/kg | |
| | | of body weight | |
| 100 μmol of Gd/kg | 11 μmol/kg of body | 11 μmol of EPO/kg | |
| of body weight | weight | of body weight | |
| 20 mmol of Gd/l of | 1.5 mmol of | F2-F | 8 |
| the complex | epothilone | 100 μmol of Gd/kg | |
| | | of body weight | |
| 100 μmol of Gd/kg | 18 μmol/kg of body | 18 μmol of EPO/kg | |
| of body weight | weight | of body weight | |
| 20 mmol of Gd/l of | 1.5 mmol of | F1-G | 9 |
| the complex | epothilone | 100 μmol of Gd/kg | |
| | | of body weight | |
| 100 μmol of Gd/kg | 11 μmol/kg of body | 11 μmol of EPO/kg | |
| of body weight | weight | of body weight | |
| 20 mmol of Gd/l of | 1.5 mmol of | F2-G | 10 |
| the complex | epothilone | 100 μmol of Gd/kg | |
| | | of body weight | |
| 100 μmol of Gd/kg | 18 μmol/kg of body | 18 μmol of EPO/kg | |
| of body weight | weight | of body weight | |
| 20 mmol of Gd/l of | 1.5 mmol of | F1-I | 11 |
| the complex | epothilone | 100 μmol of Gd/kg | |
| | | of body weight | |
| 100 μmol of Gd/kg | 11 μmol/kg of body | 11 μmol of EPO/kg | |
| of body weight | weight | of body weight | |
| 20 mmol of Gd/l of | 1.5 mmol of | F2-K | 12 |
| the complex | epothilone | 100 μmol of Gd/kg | |
| | | of body weight | |
| 100 μmol of Gd/kg | 18 μmol/kg of body | 18 μmol of EPO/kg | |
| of body weight | weight | of body weight | |
| 20 mmol of Gd/l of | 1.5 mmol of | F1-L | 13 |
| the complex | epothilone | 100 μmol of Gd/kg | |
| | | of body weight | |
| 100 μmol of Gd/kg | 11 μmol/kg of body | 11 μmol of EPO/kg | |
| of body weight | weight | of body weight | |
| 20 mmol of Gd/l of | 1.5 mmol of | F2-L | 14 |
| the complex | epothilone | 100 μmol of Gd/kg | |
| | | of body weight | |
| 100 μmol of Gd/kg | 18 μmol/kg of body | 18 μmol of EPO/kg | |
| of body weight | weight | of body weight | |
| 20 mmol of Gd/l of | 1.5 mmol of | F2-K | 15 |
| the complex | epothilone | 100 μmol of Gd/kg | |
| | | of body weight | |
| 100 μmol of Gd/kg | 18 μmol/kg of body | 18 μmol of EPO/kg | |
| of body weight | weight | of body weight | |
| 20 mmol of Gd/l of | 1.5 mmol of | F1-N | 16 |
| the complex | epothilone | 100 μmol of Gd/kg | |
| | | of body weight | |
| 100 μmol of Gd/kg | 11 μmol/kg of body | 11 μmol of EPO/kg | |
| of body weight | weight | of body weight | |

Results of Tumor-Therapy Tests:
Tumor-Therapy Test No. 1

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at
the beginning of treatment (day 0) were normalized to 1.

| Tumor Surface | | | | | |
|---|---|---|---|---|---|
| Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.0 ± 0.1 | 1.2 ± 0.2 | 1.4 ± 0.3 | 1.5 ± 0.3 |
| (c) | 1 | 1.0 ± 0.2 | 1.0 ± 0.3 | 1.2 ± 0.2 | 1.1 ± 0.1 |

TABLE 2
Relative animal weights. The animal weights at the beginning
of treatment (day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.93 | 0.95 | 0.96 | 0.95 |
| (c) | 1 | 0.96 | 1 | 1 | 1.05 |

Tumor-Therapy Test No. 2

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at
the beginning of treatment (day 0) were normalized to 1.

| Tumor Surface | | | | | |
|---|---|---|---|---|---|
| Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.3 ± 0.1 | 1.4 ± 0.3 | 1.5 ± 0.2 |
| (c) | 1 | 1.0 ± 0.2 | 1.0 ± 0.2 | 1.2 ± 0.3 | 1.2 ± 0.2 |

TABLE 2
Relative animal weights. The animal weights at the beginning
of treatment (day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.94 | 0.97 | 0.96 | 0.96 |
| (c) | 1 | 0.96 | 1 | 1.01 | 1.05 |

Tumor-Therapy Test No. 3

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at
the beginning of treatment (day 0) were normalized to 1.

| Tumor Surface | | | | | |
|---|---|---|---|---|---|
| Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.4 ± 0.3 | 1.5 ± 0.3 | 1.5 ± 0.2 |
| (c) | 1 | 1.0 ± 0.2 | 1.0 ± 0.1 | 1.1 ± 0.4 | 1.1 ± 0.2 |

TABLE 2
Relative animal weights. The animal weights at the beginning
of treatment (day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.94 | 0.97 | 0.96 | 0.96 |
| (c) | 1 | 0.95 | 1 | 1.02 | 1.05 |

Tumor-Therapy Test No. 4

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at
the beginning of treatment (day 0) were normalized to 1.

| Tumor Surface | | | | | |
|---|---|---|---|---|---|
| Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.3 ± 0.3 | 1.6 ± 0.3 | 1.5 ± 0.2 |
| (c) | 1 | 1.0 ± 0.1 | 1.0 ± 0.3 | 1.2 ± 0.2 | 1.1 ± 0.2 |

TABLE 2
Relative animal weights. The animal weights at the beginning
of treatment (day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.95 | 0.97 | 0.97 | 0.96 |
| (c) | 1 | 0.95 | 1.01 | 1 | 1.04 |

Tumor-Therapy Test No. 5

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at
the beginning of treatment (day 0) were normalized to 1.

| Tumor Surface | | | | | |
|---|---|---|---|---|---|
| Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.4 ± 0.2 | 1.5 ± 0.2 | 1.5 ± 0.4 |
| (c) | 1 | 1.0 ± 0.2 | 1.0 ± 0.3 | 1.2 ± 0.2 | 1.2 ± 0.2 |

TABLE 2
Relative animal weights. The animal weights at the beginning
of treatment (day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.94 | 0.96 | 0.97 | 0.96 |
| (c) | 1 | 0.95 | 1.01 | 1.02 | 1.03 |

Tumor-Therapy Test No. 6

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas
at the beginning of treatment (day 0) were normalized to 1.

| Tumor Surface | | | | | |
|---|---|---|---|---|---|
| Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.2 ± 0.1 | 1.5 ± 0.2 | 1.5 ± 0.3 |
| (c) | 1 | 1.0 ± 0.2 | 1.0 ± 0.1 | 1.2 ± 0.4 | 1.1 ± 0.2 |

TABLE 2
Relative animal weights. The animal weights at the
beginning of treatment (day 0, about 30-35 g)
were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.93 | 0.97 | 0.96 | 0.96 |
| (c) | 1 | 0.94 | 1 | 1.02 | 1.03 |

Tumor-Therapy Test No. 7

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at the
beginning of treatment (day 0) were normalized to 1.

| Tumor Surface | | | | | |
|---|---|---|---|---|---|
| Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.3 ± 0.2 | 1.5 ± 0.3 | 1.5 ± 0.4 |
| (c) | 1 | 1.0 ± 0.2 | 1.0 ± 0.3 | 1.2 ± 0.4 | 1.1 ± 0.2 |

TABLE 2
Relative animal weights. The animal weights at the beginning
of treatment (day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.94 | 0.97 | 0.97 | 0.96 |
| (c) | 1 | 0.95 | 1 | 1 | 1.05 |

Tumor-Therapy Test No. 8

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at the
beginning of treatment (day 0) were normalized to 1.

| Tumor Surface | | | | | |
|---|---|---|---|---|---|
| Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.2 ± 0.2 | 1.4 ± 0.1 | 1.5 ± 0.2 |
| (c) | 1 | 1.0 ± 0.2 | 1.0 ± 0.2 | 1.1 ± 0.3 | 1.1 ± 0.1 |

TABLE 2
Relative animal weights. The animal weights at the beginning
of treatment (day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.92 | 0.93 | 0.97 | 0.99 |
| (c) | 1 | 0.93 | 0.98 | 1.01 | 1.03 |

Tumor-Therapy Test No. 9

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at the
beginning of treatment (day 0) were normalized to 1.

| Tumor Surface | | | | | |
|---|---|---|---|---|---|
| Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.3 ± 0.1 | 1.5 ± 0.3 | 1.6 ± 0.2 |
| (c) | 1 | 1.0 ± 0.2 | 1.0 ± 0.3 | 1.2 ± 0.2 | 1.1 ± 0.1 |

TABLE 2
Relative animal weights. The animal weights at the beginning
of treatment (day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.94 | 0.97 | 0.95 | 0.96 |
| (c) | 1 | 0.95 | 1.01 | 1 | 1.02 |

Tumor-Therapy Test No. 10

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at the
beginning of treatment (day 0) were normalized to 1.

| Tumor Surface | | | | | |
|---|---|---|---|---|---|
| Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.3 ± 0.4 | 1.5 ± 0.3 | 1.6 ± 0.2 |
| (c) | 1 | 1.0 ± 0.1 | 1.0 ± 0.3 | 1.2 ± 0.1 | 1.1 ± 0.2 |

TABLE 2
Relative animal weights. The animal weights at the beginning
of treatment (day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.945 | 0.97 | 0.97 | 0.98 |
| (c) | 1 | 0.95 | 1.01 | 1.02 | 1.05 |

Tumor-Therapy Test No. 11

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at the beginning of
treatment (day 0) were normalized to 1.

| Tumor | | | | | |
|---|---|---|---|---|---|
| Surface Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.2 ± 0.1 | 1.3 ± 0.3 | 1.4 ± 0.3 | 1.5 ± 0.1 |
| (c) | 1 | 1.1 ± 0.2 | 1.1 ± 0.3 | 1.2 ± 0.1 | 1.0 ± 0.2 |

TABLE 2
Relative animal weights. The animal weights at the beginning of
treatment (day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.93 | 0.97 | 0.97 | 0.98 |
| (c) | 1 | 0.95 | 1 | 1.03 | 1.05 |

Tumor-Therapy Test No. 12

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at the beginning of
treatment (day 0) were normalized to 1.

| Tumor | | | | | |
|---|---|---|---|---|---|
| Surface Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.3 ± 0.3 | 1.5 ± 0.2 | 1.6 ± 0.1 |
| (c) | 1 | 1.0 ± 0.1 | 1.0 ± 0.3 | 1.2 ± 0.3 | 1.1 ± 0.2 |

TABLE 2
Relative animal weights. The animal weights at the beginning of treatment
(day 0, about 30-35 g) were normalized to 1.

| | | | | | |
|---|---|---|---|---|---|
| Animal Weights | Day 0 | Day 5 | Day 11 | Day 15 | Day 20 |

| | | | | | |
|---|---|---|---|---|---|
| (a) | 1 | 0.99 | 1 | 1 | 0.99 |
| (b) | 1 | 0.95 | 0.96 | 0.97 | 0.97 |
| (c) | 1 | 0.95 | 1.01 | 1 | 1.02 |

Tumor-Therapy Test No. 13

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at the beginning of
treatment (day 0) were normalized to 1.

| Tumor | | | | | |
|---|---|---|---|---|---|
| Surface Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.3 ± 0.1 | 1.5 ± 0.2 | 1.5 ± 0.2 |
| (c) | 1 | 1.0 ± 0.2 | 1.1 ± 0.2 | 1.2 ± 0.3 | 1.1 ± 0.1 |

TABLE 2
Relative animal weights. The animal weights at the beginning of treatment
(day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.93 | 0.96 | 0.97 | 0.98 |
| (c) | 1 | 0.95 | 1.01 | 1.02 | 1.03 |

Tumor-Therapy Test No. 14

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at the beginning of
treatment (day 0) were normalized to 1.

| Tumor | | | | | |
|---|---|---|---|---|---|
| Surface Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.3 ± 0.2 | 1.5 ± 0.2 | 1.5 ± 0.1 |
| (c) | 1 | 1.0 ± 0.2 | 1.0 ± 0.1 | 1.2 ± 0.3 | 1.1 ± 0.2 |

TABLE 2
Relative animal weights. The animal weights at the beginning of treatment
(day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.94 | 0.95 | 0.97 | 0.96 |
| (c) | 1 | 0.95 | 1 | 1.02 | 1.05 |

Tumor-Therapy Test No. 15

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at the beginning of
treatment (day 0) were normalized to 1.

| Tumor | | | | | |
|---|---|---|---|---|---|
| Surface Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.3 ± 0.1 | 1.5 ± 0.2 | 1.5 ± 0.2 |
| (c) | 1 | 1.0 ± 0.2 | 1.0 ± 0.3 | 1.1 ± 0.3 | 1.1 ± 0.2 |

TABLE 2
Relative animal weights. The animal weights at the beginning of treatment
(day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.95 | 0.96 | 0.97 | 0.97 |
| (c) | 1 | 0.95 | 1 | 1.02 | 1.04 |

Tumor-Therapy Test No. 16

In group (c), the tumor growth was inhibited over about 20 days compared to control groups (a) and (b) (Tab. 1). The relative animal weights, with the exception of group (a), indicated a small weight loss in the first days after substance administration (Tab. 2).

TABLE 1
Relative tumor surface areas. The tumor surface areas at the
beginning of treatment (day 0) were normalized to 1.

| Tumor Surface | | | | | |
|---|---|---|---|---|---|
| Area | Day 0 | Day 5 | Day 11 | Day 15 | Day 20 |
| (a) | 1 | 1.3 ± 0.2 | 1.6 ± 0.3 | 1.8 ± 0.3 | 1.8 ± 0.1 |
| (b) | 1 | 1.1 ± 0.1 | 1.2 ± 0.2 | 1.5 ± 0.3 | 1.6 ± 0.4 |
| (c) | 1 | 1.0 ± 0.2 | 1.0 ± 0.1 | 1.2 ± 0.2 | 1.1 ± 0.2 |

TABLE 2
Relative animal weights. The animal weights at the beginning
of treatment (day 0, about 30-35 g) were normalized to 1.

| Animal Weights | Day 0 | Day 5 | Day 11 | Day 15 | Day 20 |
|---|---|---|---|---|---|
| (a) | 1 | 0.99 | 1 | 1 | 0.99 |
| (b) | 1 | 0.94 | 0.97 | 0.98 | 0.96 |
| (c) | 1 | 0.96 | 1.01 | 1 | 1.04 |

Summary of the Results of the Tumor-Therapy Tests

It is shown that these new inventive formulations have definite advantages compared to the prior art (EPO alone), i.e., produce a significant delay of tumor growth. This advantage is statistically significant (p<0.001, t-test).

Also, the higher doses are very well tolerated, which corresponds to good compatibility of these formulations.

In particular, the substances according to the invention are extremely well suited for intravenous administration. The intravenous administration of low-osmolar solutions causes only slight discomfort to the patient's body.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 102005008309.9, filed Feb. 17, 2005, and U.S. Provisional Application Ser. No. 60/657,694, filed Mar. 3, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Pharmaceutical agents for intravenous administration that contain a) at least one fluoroalkyl-containing metal complex, which contains an ion of the elements of atomic numbers 20-29, 39, 42, 44, 57-70 or 83 as a metal and which has a critical micelle-formation concentration of <10⁻³ mol/l and a hydrodynamic micelle diameter (2 Rh) of >1 nm, and b) at least one natural or synthetic epothilone or epothilone derivative, optionally with the additives that are commonly used in galenicals.

2. Pharmaceutical agents according to claim 1, characterized in that metal complexes are used whose micelle-formation concentration is <10⁻⁴ mol/l.

3. Pharmaceutical agents according to claim 1, wherein metal complexes are used whose micelle-formation concentration is <10⁻⁵.

4. Pharmaceutical agents according to claim 1, wherein metal complexes are used whose hydrodynamic micelle diameter is ≧2 nm.

5. Pharmaceutical agents according to claim 1, wherein metal complexes are used whose hydrodynamic micelle diameter is ≧3 nm.

6. Pharmaceutical agents according to claim 1, wherein the fluoroalkyl-containing metal complex contains a paramagnetic ion of the elements of atomic numbers 21-29, 42, 44, or 58-70 as a metal.

7. Pharmaceutical agents according to claim 1, wherein as fluoroalkyl-containing metal complexes, the compounds of general formula I
R^{F}-L-K I
in which
R^{F} is a fluorinated, straight-chain or branched carbon chain with formula —CₙF₂ₙE, in which
E represents a terminal fluorine, chlorine, bromine, iodine or hydrogen atom, and n stands for numbers 4-30,
L means a direct bond, a methylene group, an —NHCO group, a group 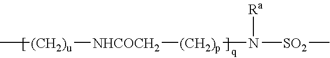
whereby p means the numbers 0 to 10, q and u, independently of one another, mean the number 0 or 1, and
R^{a} is a hydrogen atom, a methyl group, a —CH₂—OH group, a —CH₂—CO₂H group or a C₂-C₁₅ chain, which optionally is interrupted by 1 to 3 oxygen atoms, 1 to 2 C(O) groups or an optionally substituted aryl group and/or is substituted with 1 to 4 hydroxyl groups, 1 to 2 C₁-C₄-alkoxy groups, 1 to 2 carboxy groups, or a group —SO₃H—,
or a straight-chain, branched, saturated or unsaturated C₂-C₃₀-carbon chain, which optionally contains 1 to 10 oxygen atoms, 1 to 3 —NR^{a} groups, 1 to 2 sulfur atoms, a piperazine, a —CONR^{a} group, an —NR^{a}CO group, an —SO₂ group, an —NR^{a}—CO₂ group, 1 to 2 —CO groups, a group —CO—NH—T-N(R^{a})—SO₂—R^{F} or 1 to 2 optionally substituted aryls and/or is interrupted by these groups, and/or optionally is substituted with 1 to 3 —OR^{a} groups, 1 to 2 oxo groups, 1 to 2 —NH—COR^{a} groups, 1 to 2 —CONHR^{a} groups, 1 to 2 —(CH₂)ₚ—CO₂H groups, or 1 to 2 —(CH₂)ₚ—(O)_{q}—CH₂CH₂—R^{F} groups,
whereby
R^{a}, R^{F} and p and q have the above-indicated meanings, and
T means a C₂-C₁₀-chain, which optionally is interrupted by 1 to 2 oxygen atoms or 1 to 2 —NHCO groups,
K stands for a metal complex or salts thereof of organic and/or inorganic bases or amino acids or amino acid amides, specifically for a complex of general formula 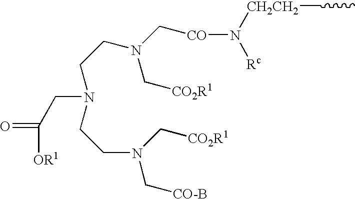
in which R^{c}, R¹ and B are independent of one another, and
R^{c} has the meaning of R^{a} or means —(CH₂)ₘ-L-R^{F}, whereby m is 0, 1 or 2, and L and R^{F} have the above-mentioned meaning,
R¹, independently of one another, mean a hydrogen atom or a metal ion equivalent of atomic numbers 20-29, 39, 42, 44, 58-70 or 83, whereby at least two R¹ mean a metal ion equivalent,
B means —OR¹ or 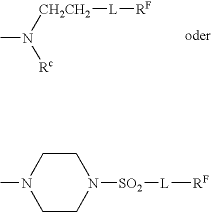
[oder=or]
whereby R¹, L, R^{F} and R^{c} have the above-mentioned meanings, or for a complex of general formula III 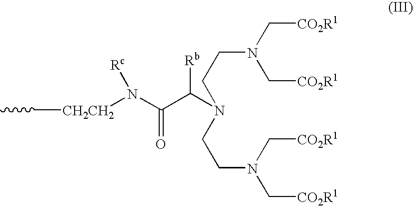
in which R^{c} und R¹ have the above-mentioned meanings, and R^{b} has the meaning of R^{a}
or
for a complex of general formula IV 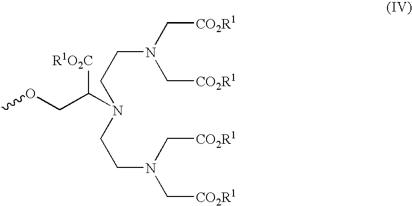
in which R¹ has the above-mentioned meaning,
or
for a complex of general formula V 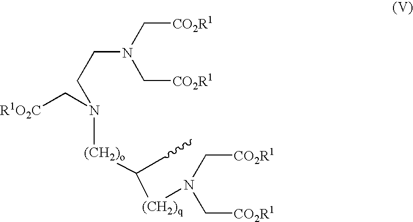
in which R¹ has the above-mentioned meaning, and o and q stand for number O or 1, and yields the sum o+q=1,
or
for a complex of general formula VI 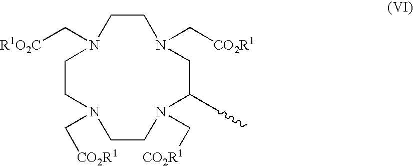
in which R¹ has the above-mentioned meaning
or
for a complex of general formula VII 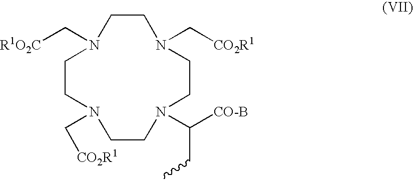
in which R¹ and B have the above-mentioned meanings
or
for a complex of general formula VIII 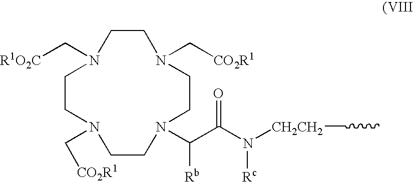
in which R^{c} and R¹ have the above-mentioned meanings, and
R^{b} has the above-mentioned meaning of R^{a}.
or
for a complex of general formula IX 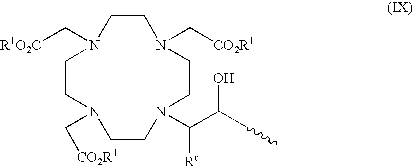
in which R^{c} and R¹ have the above-mentioned meanings,
or
for a complex of general formula X 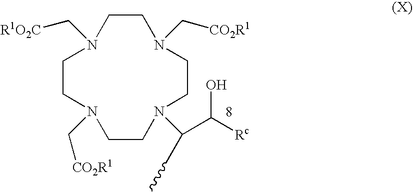
in which R^{c} and R¹ have the above-mentioned meanings,
or
for a complex of general formula XI 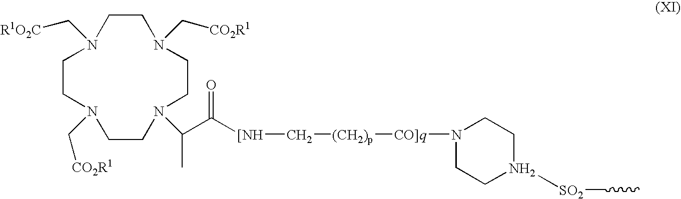
in which R¹, p and q have the above-mentioned meaning, and R^{b} has the meaning of R^{a}
or
for a complex of general formula XII 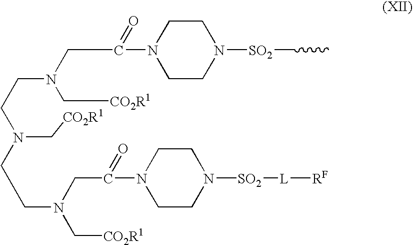
in which L, R^{F} and Z¹ have the above-mentioned meanings,
or
for a complex of general formula XIII 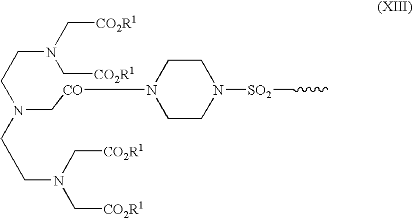
in which R¹ has the above-mentioned meaning, are used.

8. Pharmaceutical agents according to claim 7, wherein the compounds of general formula I are used, in which L stands for
α-CH₂-β
α-CH₂CH₂-β
α-(CH₂)_{S}-β s=3−15
α-CH₂—O—CH₂CH₂-β
α-CH₂—(O—CH₂—CH₂—)ₜ-β t=2−6
α-CH₂—NH—CO-β
α-CH₂—NH—CO—CH₂—N(CH₂COOH)—SO₂-β
α-CH₂—NH—CO—CH₂—N(C₂H₅)—SO₂-β
α-CH₂—NH—CO—CH₂—N(C₁₀H₂₁)—SO₂-β
α-CH₂—NH—CO—CH₂—N(C₆H₁₃)—SO₂-β
α-CH₂—NH—CO—(CH₂)₁₀—N(C₂H₅)—SO₂-β
α-CH₂—NH—CO—CH₂—N(—CH₂—C₆H₅)—SO₂-β
α-CH₂—NH—CO—CH₂—N(—CH₂—CH₂—OH)SO₂-β
α-CH₂—NHCO—(CH₂)₁₀—S—CH₂CH₂-β
α-CH₂NHCOCH₂—O—CH₂CH₂-β
α-CH₂NHCO(CH₂)₁₀—O—CH₂CH₂-β
α-CH₂-C₆H₄—O—CH₂CH₂-β
α-CH₂—O—CH₂—C(CH₂—OCH₂CH₂—C₆F₁₃)₂—CH₂—OCH₂—CH₂-β 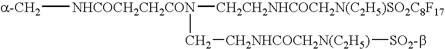
α-CH₂—O—CH₂—CH(OC₁₀H₂₁)—CH₂—O—CH₂CH₂-β
α-(CH₂NHCO)₄—CH₂O—CH₂CH₂-β
α-(CH₂NHCO)₃—CH₂O—CH₂CH₂-β
α-CH₂—OCH₂C(CH₂OH)₂—CH₂—O—CH₂CH₂-β 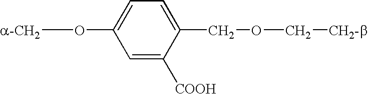
α-CH₂NHCOCH₂N(C₆H₅)—SO₂-β
α-NHCO—CH₂—CH₂-β
α-NHCO—CH₂—O—CH₂CH₂-β
α-NH—CO-β
α-NH—CO—CH₂—N(CH₂COOH)—SO₂-β
α-NH—CO—CH₂—N(C₂H₅)—SO₂-β
α-NH—CO—CH₂—N(C₁₀H₂₁)—SO₂-β
α-NH—CO—CH₂—N(C₆H₁₃)—SO₂-β
α-NH—CO—(CH₂)₁₀—N(C₂H₅)—SO₂-β
α-NH—CO—CH₂—N(—CH₂—C₆H₅)—SO₂-β
α-NH—CO—CH₂—N(—CH₂—CH₂—OH)SO₂-β
α-NH—CO—CH₂-β
α-CH₂—O—C₆H₄—O—CH₂—CH₂-β
α-CH₂—C₆H₄—O—CH₂—CH₂-β
α-N(C₂H₅)—SO₂-β
α-N(C₆H₅)—SO₂-β
α-N(C₁₀H₂₁ )—SO₂-β
α-N(C₆H₁₃)—SO₂-β
α-N(C₂H₄OH)—SO₂-β
α-N(CH₂COOH)—SO₂-β
α-N(CH₂C₆H₅)—SO₂-β
α-N—[CH(CH₂OH)₂]—SO₂-β
α-N—[CH(CH₂OH)CH(CH₂OH)]—SO₂-β
and in which a represents the binding site to metal complex K, and β represents the binding site to the fluorine radical.

9. Pharmaceutical agents according to claim 7, wherein the compounds of formula I, in which n in formula —CₙF₂ₙE stands for numbers 4-15 and/or E in this formula means a fluorine atom, are used.

10. Pharmaceutical agents according to, wherein the following compounds are used:
Gadolinium complex of 10-[1-methyl-2-oxo-3-aza-5-oxo-{4-perfluorooctylsulfonyl-piperazin-1-yl}-pentyl]-1,4,7-tris(carboxymethyl)-1,4,7,10-tetraazacyclododecane,
Gadolinium complex of 10-[2-hydroxy-4-aza-5-oxo-7-oxa-10,10,11,11,12,12, 13,13,14,14,15,15,16,16,17,17-heptadecafluoroheptadecyl]-1,4,7-tris(carboxymethyl)-1,4,7,10-tetraazacyclododecane,
Gadolinium complex of 10-[2-hydroxy-4-aza-5,9-dioxo-9-{4-perfluorooctyl)-piperazin-1-yl}-nonyl]-1,4,7-tris(carboxymethyl)-1,4,7,10-tetraazacyclododecane,
Gadolinium complex of 10-[2-hydroxy-4-aza-5-oxo-7-aza-7-(perfluorooctyl-sulfonyl)-nonyl]-1,4,7-tris(carboxymethyl)-1,4,7,10-tetraazacyclododecane,
Gadolinium complex of 10-[2-hydroxy-4-oxa-1H,1H,2H,3H,3H,5H,5H,6H,6H-perfluoro-tetradecyl]-1,4,7-tris(carboxymethyl)-1,4,7,10-tetraazacyclododecane,
Gadolinium complex of 10-[2-hydroxy-4-aza-5-oxo-7-oxa-10,10,11,11, 12,12,13,13,14,14,15,15,16,16,17,17,18,18,19,19-henicosafluoro-nonadecyl]-1,4,7-tris(carboxymethyl)-1,4,7,10-tetraazacyclododecane,
Gadolinium complex of 10-[2-hydroxy-4-aza-5-oxo-11-aza-11-(perfluoro-octylsulfonyl)-tridecyl]-1-4-7-tris(carboxymethyl)1,4,7,10-tetraazacyclododecane,
Gadolinium complex of 10-[2-hydroxy-4-aza-5-oxo-7-aza-7-(perfluorooctylsulfonyl)-8-phenyl-octyl]-1-4-7-tris(carboxymethyl)-1,4,7,10-tetraazacyclododecane.

11. Pharmaceutical agents according to claim 1, wherein as fluoroalkyl-containing metal complexes, the compounds of general formula Ia
A-R^{F} (Ia)
in which
A is a molecule portion that contains 2-6 metal complexes that are bonded directly or via a linker to a nitrogen atom of a circular skeleton chain, and
R^{F} is a fluorinated, straight-chain or branched carbon chain with formula —CₙF₂ₙE, in which
E represents a terminal fluorine, chlorine, bromine, iodine or hydrogen atom,
and n stands for numbers 4-30,
whereby molecule portion A has the following structure: 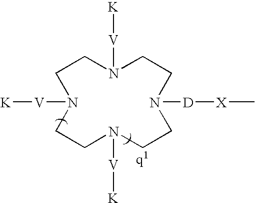
whereby
q¹ is a number 0, 1, 2 or 3,
K stands for a metal complex or salts thereof of organic and/or inorganic bases or amino acids or amino acid amides,
X is a direct bond to the fluoroalkyl group, a phenylene group or a C₁-C₁₀-alkylene chain, which optionally contains 1-15 oxygen atoms, 1-5 sulfur atoms, 1-10 carbonyl groups, 1-10 (NR^{d}) groups, 1-2 —NR^{d}—SO₂ groups, 1-10 CO—N(R^{d}) groups, 1 piperidine group, 1-3 SO₂ groups, or 1-2 phenylene groups or optionally is substituted by 1-3 radicals R^{F}, in which R^{d} stands for a hydrogen atom, a phenyl group, a benzyl group or a C₁-C₁₅-alkyl group, which optionally contains 1-2 NHCO groups, 1-2 CO groups, or 1-5 oxygen atoms and optionally is substituted by 1-5 hydroxy radicals, 1-5 methoxy radicals, 1-3 carboxy radicals, or 1-3 R^{F} radicals.
V is a direct bond or a chain of general formula IIa or IIIa: 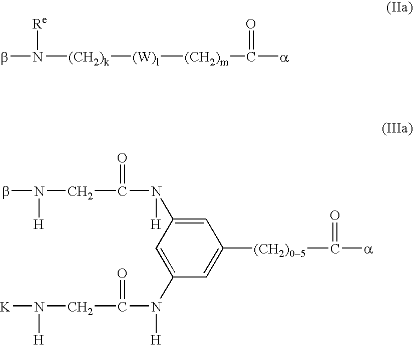
in which
R^{e} is a hydrogen atom, a phenyl group, a benzyl group or a C₁-C₇ alkyl group, which optionally is substituted with a carboxy group, a methoxy group, or a hydroxy group,
W is a direct bond, a polyglycol ether group with up to 5 glycol units or a molecule portion of general formula IVa
—CH(R^{h})— (IVa)
in which R^{h} is a C₁-C₇-carboxylic acid radical, a phenyl group, a benzyl group or a —(CH₂)₁₋₅—NH—K group,
α represents the binding to the nitrogen atom of the skeleton chain, β represents the binding to metal complex K,
and in which variables k and m stand for natural numbers between 0 and 10 and 1 stands for 0 or 1,
and whereby
D is a CO group or SO₂ group, are used.

12. Pharmaceutical agents according to claim 11, wherein the compounds of general formula Ia, in which q¹ is the number 1, are used.

13. Pharmaceutical agents according to claim 11, wherein the compounds of general formula Ia, in which molecule portion X is an alkylene chain that contains 1-10 —CH₂CH₂O groups or 1-5 —COCH₂NH groups, a direct bond or one of the following structures 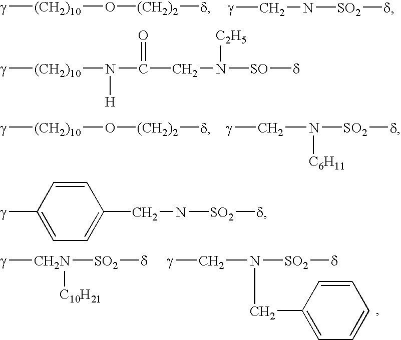
whereby
γ binds to D and δ binds to R^{F}, are used.

14. Pharmaceutical agents according to claim 11, wherein the compounds of formula Ia, in which V is a molecule portion with one of the following structures 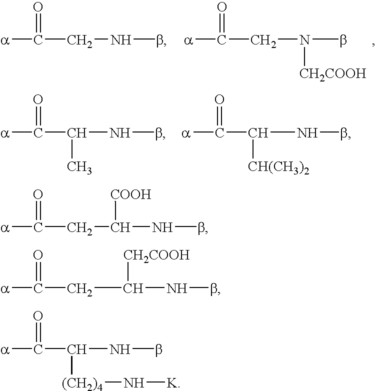
are used.

15. Pharmaceutical agents according to claim 11, wherein the compounds of general formula Ia, in which K represents a complex of general formula Va, VIa, VIIa or VIIIa 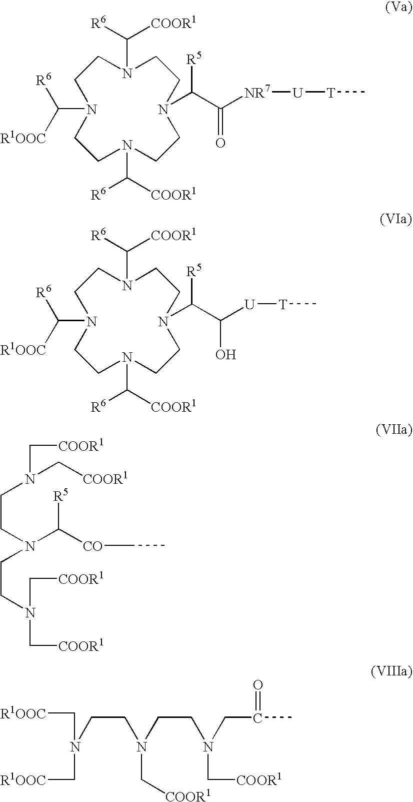
whereby
R¹, independently of one another, are a hydrogen atom or a metal ion
equivalent of the elements of atomic numbers 20-29, 39, 42, 44, 58-70 or 83, whereby at least two R¹ mean a metal ion equivalent,
R⁵ is a hydrogen atom or a straight-chain, branched, saturated or unsaturated C₁-C₃₀-alkyl chain, which optionally is substituted by 1-5 hydroxy groups, 1-3 carboxy groups or 1 phenyl group and/or optionally is interrupted by 1-10 oxygen atoms, 1 phenylene group or 1phenylenoxy group,
R⁶ is a hydrogen atom, a straight-chain or branched C₁-C₇-alkyl radical, a phenyl radical or a benzyl radical,
R⁷ is a hydrogen atom, a methyl group or an ethyl group, which optionally is substituted by a hydroxy group or a carboxy group,
U³ is a straight-chain, branched, saturated or unsaturated C₁-C₂₀-alkylene
group that optionally contains 1-5 imino groups, 1-3 phenylene groups, 1-3 phenylenoxy groups, 1-3 phenylenimino groups, 1-5 amide groups, 1-2 hydrazide groups, 1-5 carbonyl groups, 1-5 ethylenoxy groups, 1 urea group, 1 thiourea group, 1-2 carboxyalkylimino groups,
1-2 ester groups, 1-10 oxygen atoms, 1-5 sulfur atoms, and/or 1-5 nitrogen atoms and/or that optionally is substituted by 1-5 hydroxy groups, 1-2 mercapto groups, 1-5 oxo groups, 1-5 thioxo groups, 1-3 carboxy groups, 1-5 carboxyalkyl groups, 1-5 ester groups and/or 1-3 amino groups, whereby the optionally contained phenylene groups can be substituted by 1-2 carboxy groups, 1-2 sulfone groups or 1-2 hydroxy groups,
T¹ stands for a —CO-β, —NHCO-β or —NHCS—β group, whereby β represents the binding site to V,
are used.

16. Pharmaceutical agents according to claim 15, wherein the C₁-C₂₀-alkylene chain that stands for U³ contains the groups —CH₂NHCO—, —NHCOCH₂O—, —NHCOCH₂OC₆H₄—, —N(CH₂CO₂H)—, —CH₂OCH₂—, —NHCOCH₂C₆H₄—, —NHCSNHC₆H₄—, —CH₂OC₆H₄—, and —CH₂CH₂O— and/or is substituted by the groups —COOH or —CH₂COOH.

17. Pharmaceutical agents according to claim 15, wherein U³ stands for a —CH₂—, —CH₂CH₂—, —CH₂CH₂CH₂—, —C₆H₄—, —C₆H₁₀—, —CH₂C₆H₄—, —CH₂NHCOCH₂CH(CH₂CO₂H)—C₆H₄—, —CH₂NHCOCH₂OCH₂—, or —CH₂NHCOCH₂C₆H₄ group.

18. Pharmaceutical agents according to claim 11, wherein the compounds of general formula Ia, in which K has one of the following structures: 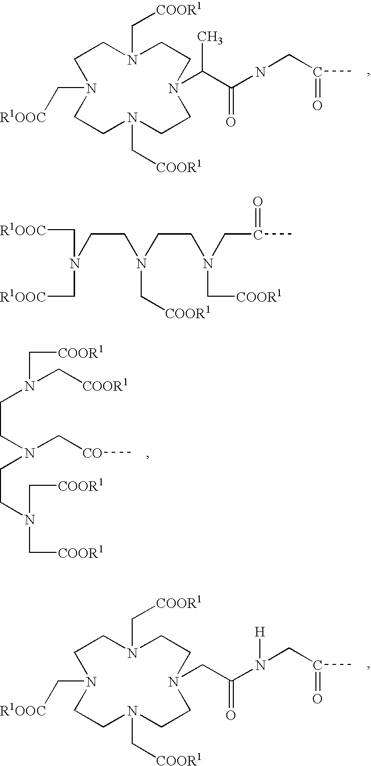
are used.

19. Pharmaceutical agents according to claim 11, 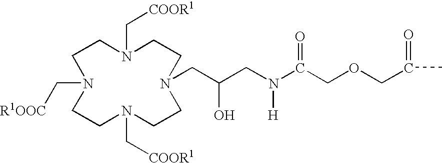
wherein the compounds of general formula Ia, in which the fluoroalkyl chain R^{F} is —C₆F₁₃,
—C₈F₁₇, —C₁₀F₂₁ or —C₁₂F₂₅, are used.

20. Pharmaceutical agents according to claim 11, wherein the gadolinium complex of 1,4,7-tris{1,4,7-tris(N-(carboxylatomethyl)-10-[N-1-methyl-3,6-diaza-2,5,8-trioxooctane-1,8-diyl)]-1,4,7,10-tetraazacyclododecane, Gd complex}-10-[N-2H, 2H, 4H, 4H, 5H, 5H-3-oxa-perfluoro-tridecanoyl]-1,4,7,10-tetraazacyclododecane is used.

21. Pharmaceutical agents according to claim 1, wherein as fluoroalkyl-containing metal complexes, the compounds of general formula Ib 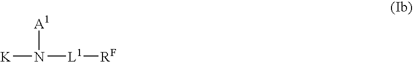
in which
K means a metal complex of general formula IIb 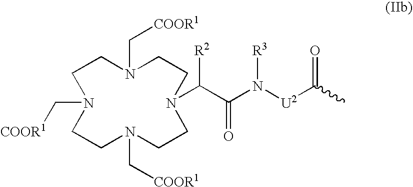
whereby R¹ stands for a hydrogen atom or a metal ion equivalent of atomic numbers 20-29, 39, 42, 44, 58-70 or 83, whereby at least two R¹mean a metal ion equivalent,
R² and R³ stand for a hydrogen atom, a C₁-C₇-alkyl group, a benzyl group, a phenyl group, —CH₂OH or —CH₂—OCH₃,
U² stands for the radical L¹, but L¹ and U², independently of one another, can be the same or different,
A¹ means a hydrogen atom, a straight-chain or branched C₁-C₃₀-alkyl group, which optionally is interrupted by 1-15 oxygen atoms, and/or optionally is substituted with 1-10 hydroxy groups, 1-2 COOH groups, a phenyl group, a benzyl group and/or 1-5 —OR^{g} groups, with R^{g} in the meaning of a hydrogen atom or a C₁-C₇-alkyl radical, or -L¹-R^{F},
L¹ means a straight-chain or branched C₁-C₃₀-alkylene group, which optionally is interrupted by 1-10 oxygen atoms, 1-5 —NH—CO groups, 1-5 —CO—NH groups, by a phenylene group that optionally is substituted by a COOH group; 1-3 sulfur atoms, 1-2 —N(B¹)—SO₂— groups, and/or 1-2 —SO₂—N(B¹) groups with B¹ in the meaning of A¹, an NHCO group, a CONH group, an N(B¹)—SO₂— group, or an —SO₂—N(B¹)— group and/or optionally is substituted with the radical R^{F}, and
R^{F} means a straight-chain or branched, fluorinated alkyl radical of formula CₙF₂ₙE,
whereby n stands for numbers 4-30, and
E stands for a terminal fluorine atom, chlorine atom, bromine atom, iodine atom or a hydrogen atom,
and optionally present acid groups optionally can be present as salts of organic and/or inorganic bases or amino acids or amino acid amides, are used.

22. Pharmaceutical agents according to claim 21, wherein the compounds of general formula Ib, in which R², R³ and R^{g}, independently of one another, mean hydrogen or a C₁-C₄-alkyl group, are used.

23. Pharmaceutical agents according to claim 21, wherein the compounds of general formula Ib, in which A¹ means hydrogen, a C₁-C₁₅-alkyl radical, the radicals C₂H₄—O—CH₃, C₃H₆—O—CH₃, C₂H₄—O—(C₂H₄—O)ₜ—C₂H₄—OH, C₂H₄—O—(C₂H₄—O)ₜ—C₂H₄—OCH₃, C₂H₄OH, C₃H₆OH, C₄H₈OH, C₅H₁₀OH, C₆H₁₂OH, C₇H₁₄OH, CH(OH)CH₂OH, CH(OH)CH(OH)CH₂OH, CH₂[CH(OH)]ᵤ¹CH₂OH, CH[CH₂(OH)]CH(OH)CH₂OH, C₂H₄CH(OH)CH₂OH, (CH₂)ₛCOOH, C₂H₄—O—(C₂H₄—O)ₜ—CH₂COOH or C₂H₄—O—(C₂H₄—O)ₜ—C₂H₄—CₙF₂ₙE,
whereby
s stands for integers 1 to 15,
t stands for integers 0 to 13,
u¹ stands for integers 1 to 10,
n stands for integers 4 to 20, and
E stands for a hydrogen atom, fluorine atom, chlorine atom, bromine atom or iodine atom, as well as, if possible, their branched isomers,
are used.

24. Pharmaceutical agents according to claim 21, wherein the compounds of general formula Ib, in which A¹ means hydrogen, C₁-C₁₀-alkyl, C₂H₄—O—CH₃, C₃H₆—O—CH₃, C₂H₄—O—(C₂H₄—O)—C₂H₄—OH, C₂H₄—O—(C₂H₄—O)ₓ—C₂H₄—OCH₃, C₂H₄OH, C₃H₆OH, CH₂[CH(OH)]_{y}CH₂OH, CH[CH₂(OH)]CH(OH)CH₂OH, (CH₂)_{w}COOH, C₂H₄—O—(C₂H₄—O)ₓ—CH₂COOH, C₂H₄—O—(C₂H₄—O)ₓ—C₂H₄—CₙF₂ₙE,
whereby
x stands for integers 0 to 5,
y stands for integers 1 to 6,
w stands for integers 1 to 10,
n stands for integers 4 to 15 and
E stands for a fluorine atom, as well as, if possible, their branched isomers,
are used.

25. Pharmaceutical agents according to claim 21, wherein the compounds of general formula Ib, in which L¹ means
α-(CH₂)_{S}-β
α-CH₂—CH₂—(O—CH₂—CH₂—)_{y}⁻β
α-CH₂—(O—CH₂—CH₂—)_{y}⁻ [symbol] β
α-CH₂—NH—CO-β
α-CH₂—CH₂—NH—SO₂-β
α-CH₂—NH—CO—CH₂—N(CH₂COOH)—SO₂-β
α-CH₂—NH—CO—CH₂—N(C₂H₅)—SO₂-β
α-CH₂—NH—CO—CH₂—N(C₁₀H₂₁)—SO₂-β
α-CH₂—NH—CO—CH₂—N(C₆H₁₃)—SO₂-β
α-CH₂—NH—CO—(CH₂)₁₀—N(C₂H₅)—SO₂-β
α-CH₂—NH—CO—CH₂—N(—CH₂—C₆H₅)—SO₂-β
α-CH₂—NH—CO—CH₂—N(—CH₂—CH₂—OH)SO₂-β
α-CH₂—NHCO—(CH₂)₁₀—S—CH₂CH₂-β
α-CH₂NHCOCH₂—O—CH₂CH₂-β
α-CH₂—CH₂NHCOCH₂—O—CH₂CH₂-β
α-CH₂—(CH₂—CH₂—O)ₜ—(CH₂)₃NHCO—CH₂—O—CH₂CH₂-β
α-CH₂NHCO(CH₂)₁₀—O—CH₂CH₂-β
α-CH₂CH₂NHCO(CH₂)₁₀—O—CH₂CH₂-β
α-CH₂-C₆H₄—O—CH₂CH₂-β whereby the phenylene group 1,4 or 1,3 is linked
α-CH₂—O—CH₂—C(CH₂—OCH₂CH₂—C₆F₁₃)₂—CH₂—OCH₂—CH₂-β
α-CH₂—NHCOCH₂CH₂CON—CH₂CH₂NHCOCH₂N(C₂H₅)SO₂C₈F₁₇β
α-CH₂—CH₂NHCOCH₂N(C₂H₅)—SO₂-β
α-CH₂—O—CH₂—CH(OC₁₀H₂₁)—CH₂—O—CH₂CH₂-β
α-(CH₂NHCO)₄—CH₂O—CH₂CH₂-β
α-(CH₂NHCO)₃—CH₂O—CH₂CH₂-β
α-CH₂—OCH₂C(CH₂OH)₂—CH₂—O—CH₂CH₂-β 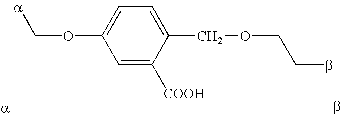
α-CH₂NHCOCH₂N(C₆H₅)—SO₂-β
α-NHCO—CH₂—CH₂-β
α-NHCO—CH₂—O—CH₂CH₂-β
α-NH—CO-β
α-NH—CO—CH₂—N(CH₂COOH)—SO₂-β
α-NH—CO—CH₂—N(C₂H₅)—SO₂-β
α-NH—CO—CH₂—N(C₁₀H₂₁)—SO₂-β
α-NH—CO—CH₂—N(C₆H₁₃)—SO₂-β
α-NH—CO—(CH₂)₁₀—N(C₂H₅)—SO₂-β
α-NH—CO—CH₂—N(—CH₂—C₆H₅)—SO₂-β
α-NH—CO—CH₂—N(—CH₂—CH₂—OH)SO₂-β
α-NH—CO—CH₂-β
α-CH₂—O—C₆H₄—O—CH₂—CH₂-β
α-CH₂—C₆H₄—O—CH₂—CH₂-β
α-N(C₂H₅)—SO₂-β
α-N(C₆H₅)—SO₂-β
α-N(C₁₀H₂₁)—SO₂-β
α-N(C₆H₁₃)—SO₂-β
α-N(C₂H₄OH)—SO₂-β
α-N(CH₂COOH)—SO₂-β
α-N(CH₂C₆H₅)—SO₂-β
α-N—[CH(CH₂OH)₂]—SO₂-β
α-N—[CH(CH₂OH)CH(OH)(CH₂OH)]—SO₂-β,
whereby
s stands for integers 1 to 15, and
y stands for integers 1 to 6,
are used.

26. Pharmaceutical agents according to claim 21, wherein the compounds of general formula Ib, in which L¹ means
α-CH₂—O—CH₂CH₂-β
α-CH₂—CH₂—(O—CH₂—CH₂—)_{y}⁻β
α-CH₂—(O—CH₂—CH₂—)_{y}⁻ [symbol] [symbol] β
α-CH₂—CH₂—NH—SO₂- [symbol] [symbol] β, Example 10
α-CH₂NHCOCH₂—O—CH₂CH₂- [symbol] [symbol] β
α-CH₂—CH₂NHCOCH₂—O—CH₂CH₂-β
α-CH₂—(CH₂—CH₂—O)_{y}—(CH₂)₃NHCO—CH₂—O—CH₂CH₂- [symbol] [symbol] β
α-CH₂NHCO(CH₂)₁₀—O—CH₂CH₂- [symbol] [symbol] β
α-CH₂CH₂NHCO(CH₂)₁₀—O—CH₂CH₂- [symbol] [symbol] β
α-CH₂—O—CH₂—CH(OC₁₀H₂₁)—CH₂—O—CH₂CH₂-β
α-CH₂—O—C₆H₄—O—CH₂—CH₂- [symbol] [symbol] β or
α-CH₂—C₆H₄—O—CH₂—CH₂-β,
whereby
y stands for integers 1 to 6,
are used.

27. Pharmaceutical agents according to claim 21, wherein the compounds of general formula Ib, in which R^{F} means a straight-chain or branched perfluorinated alkyl radical of formula CₙF₂ₙE, are used, whereby n stands for numbers 4 to 15, and E stands for a terminal fluorine atom.

28. Pharmaceutical agents according to claim 21, wherein the following compounds are used:
1,4,7-Tris(carboxylatomethyl)-10-(3-aza-4-oxo-hexan-5-ylic)-acid-N-(2,3-dihydroxypropyl)-N-(1H, 1H, 2H, 2H, 4H, 4H, 5H, 5H-3-oxa)-perfluorotridecyl)-amide]-1,4,7,10-tetraazacyclododecane, gadolinium complex
1,4,7-Tris(carboxylatomethyl)-10-{(3-aza-4-oxo-hexan-5-ylic)acid-N-(3,6,9,12,15-pentaoxa)-hexadecyl)-(1H, 1H, 2H, 2H, 4H, 4H, 5H, 5H-3-oxa)-perfluorotridecyl]-amide}-1,4,7,10-tetraazacyclododecane, gadolinium complex
1,4,7-Tris(carboxylatomethyl)-10-{(3-aza-4-oxo-hexan-5-ylic)-acid-N-5-hydroxy-3-oxa-pentyl)-N-(1H, 1H, 2H, 2H, 4H, 4H, 5H, 5H-3-oxa)-perfluorotridecyl]-amide}-1,4,7,10-tetraazacyclododecane, gadolinium complex
1,4,7-Tris(carboxylatomethyl-10-{(3-aza-4-oxo-hexan-5-ylic)-acid-[N-3,6,9,15-tetraoxa-12-aza-15-oxo-C₁₇-C₂₆-hepta-decafluoro)hexacosyl]-amide}-1,4,7,10-tetraazacyclododecane, gadolinium complex
1,4,7-Tris(carboxylatomethyl)-10-[(3-aza-4-oxo-hexan-5-ylic]-acid-N-(2-methoxy-ethyl)-N-(1H, 1H, 2H, 2H, 4H, 4H, 5H, 5H-3-oxa)-perfluorotridecyl]-amide}-1,4,7,10-tetraazacyclododecane, gadolinium complex.

29. Pharmaceutical agents according to claim 1, wherein as fluoroalkyl-containing metal complexes, the compounds with sugar radicals of general formula Ic 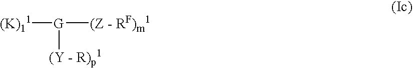
in which
R represents a monosaccharide or oligosaccharide radical that is bonded via the 1-OH— or 1-SH-position,
R^{F} is a fluorinated, straight-chain or branched carbon chain with formula —CₙF₂ₙE, in which E represents a terminal fluorine, chlorine, bromine, iodine or hydrogen atom, and n stands for numbers 4-30,
K stands for a metal complex of general formula IIc, 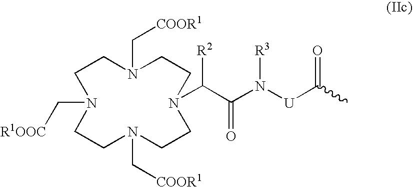
in which
R¹ means a hydrogen atom or a metal ion equivalent of atomic numbers 20-29, 39, 42, 44, 58-70 or 83, provided that at least two R¹ stand for metal ion equivalents,
R² and R³, independently of one another, represent hydrogen, C₁-C₇-alkyl, benzyl, phenyl, —CH₂OH or —CH₂OCH₃, and
U represents —C₆H₄—O—CH₂-ω-, —(CH₂)₁₋₅-ω, a phenylene group, —CH₂—NHCO—CH₂—CH(CH₂COOH)—C₆H₄-ω-, —C₆H₄—(OCH₂CH₂)₀₋₁—N(CH₂COOH)—CH₂-ω or a C₁-C₁₂-alkylene group or (CH₂)₇₋₁₂—C₆H₄—O group that optionally is interrupted by one or more oxygen atoms, 1 to 3 —NHCO groups, or 1 to 3 —CONH groups and/or substituted by 1 to 3 —(CH₂)₀₋₅COOH groups, whereby ω stands for the binding site to —CO—,
or
of general formula IIIc 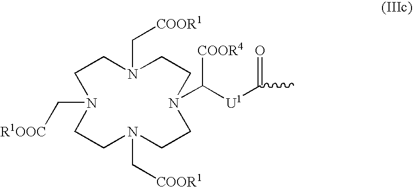
in which R¹ has the above-mentioned meaning, R⁴ represents hydrogen or a metal ion equivalent that is mentioned under R¹ and U¹ represents —C₆H₄—O—CH₂-ω-, whereby ω means the binding site to —CO—
or of general formula IVc 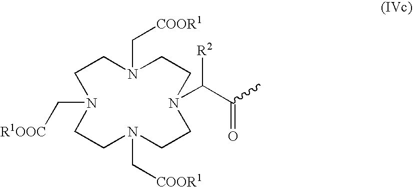
in which R¹ and R² have the above-mentioned meaning
or of general formula VcA or VcB 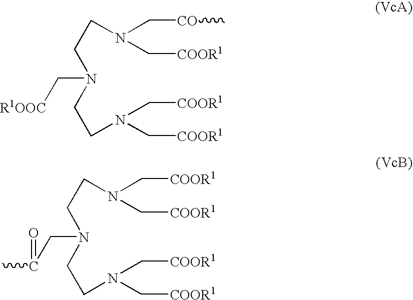
in which R¹ has the above-mentioned meaning,
or of general formula VIc 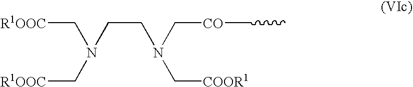
in which R¹ has the above-mentioned meaning,
or of general formula VIIc 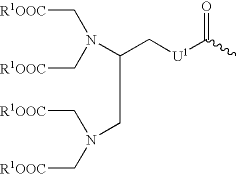
in which R¹ has the above-mentioned meaning, and
U¹ represents —C₆H₄—O—CH₂-ω-, whereby ω means the binding site to —CO— or of general formula VIIIc 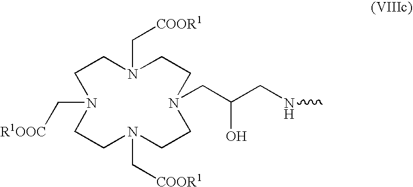
in which R¹ has the above-mentioned meaning,
and free acid groups that are optionally present in radical K optionally can be present as salts of organic and/or inorganic bases or amino acids or amino acid amides,
G, if K means metal complexes IIc to VIIc, represents a radical that is functionalized in at least three places and that is selected from radicals a) to j) below 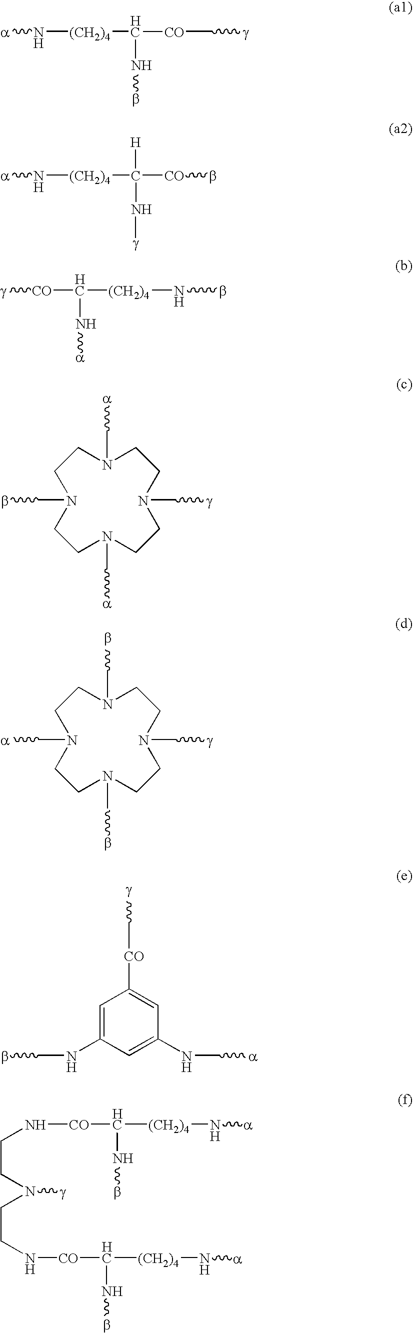 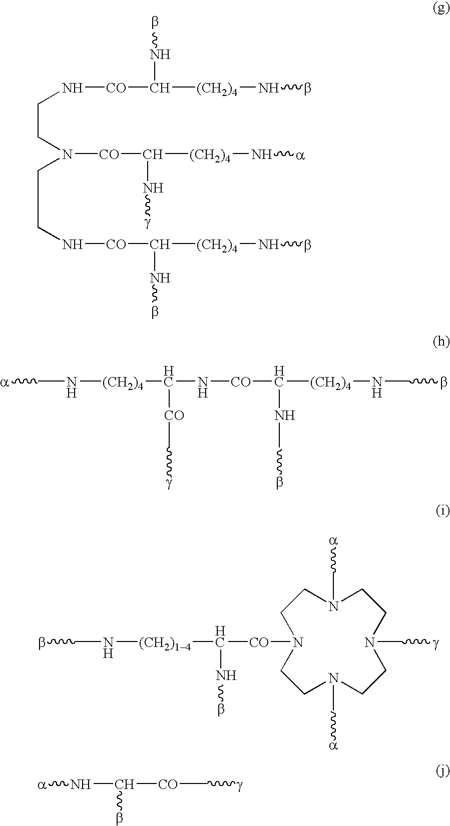
and
G, if K means metal complex VIIIc, represents a radical that is functionalized in at least three places and that is selected from k) or l), 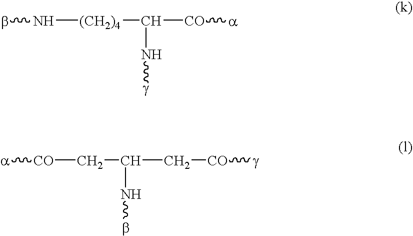
whereby α means the binding site of G to complex K, β is the binding site of G to radical Y, and γ represents the binding site of G to radical Z
Y means —CH₂—, δ-(CH₂)₍₁₋₅₎CO-β, β-(CH₂)₁₋₅CO-δ, δ-CH₂—CHOH—CO-β or δ-CH(CHOH—CH₂OH)—CHOH—CHOH—CO-β, whereby δ represents the binding site to sugar radical R, and β is the binding site to radical G
Z stands for 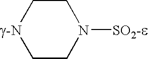
γ-COCH₂—N(C₂H₅)—SO₂-ε,
γ-COCH₂—O—(CH₂)₂—SO₂-ε, 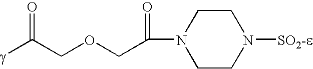
or
δ-NHCH₂CH₂—O—CH₂CH₂-ε
whereby γ represents the binding site of Z to radical G, and ε means the
binding site of Z to fluorinated radical R^{F}
and
l¹, m¹, independently of one another, mean integer 1 or 2, and
p means integers 1 to 4,
are used.

30. Pharmaceutical agents according to claim 29, wherein the compounds of general formula Ic, in which R represents a monosaccharide radical with 5 to 6 C atoms or its deoxy compound, preferably glucose, mannose or galactose, are used.

31. Pharmaceutical agents according to claim 29, wherein the compounds of general formula Ic, in which R² and R³, independently of one another, mean hydrogen or C₁-C₄-alkyl and/or E in formula —CₙF₂ₙE means a fluorine atom, are used.

32. Pharmaceutical agents according to claim 29, wherein the compounds of general formula Ic, in which G represents lysine radical (a) or (b), are used.

33. Pharmaceutical agents according to claim 29, wherein the compounds of general formula Ic, in which Z means 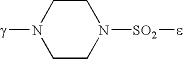
whereby γ represents the binding site of Z to radical G, and ω means the binding site of Z to fluorinated radical R^{F} and/or Y means δ-CH₂CO-β, whereby δ represents the binding site to sugar radical R and β represents the binding site to radical G, are used.

34. Pharmaceutical agents according to claim 29, wherein the compounds of general formula Ic, in which U in the metal complex K represents —CH₂— or —C₆H₄—O—CH₂-ω, whereby ω stands for the binding site to —CO—, are used.

35. Pharmaceutical agents according to claim 29, wherein the gadolinium complex of 6-N-[1,4,7-tris(carboxylatomethyl)-1,4,7,10-tetraazacyclododecane-10-N-(pentanoyl-3-aza-4-oxo-5-methyl-5-yl)]-2-N-[1-O-α-D-carbonylmethyl-mannopyranose]-L-lysine-[1-(4-perfluorooctylsulfonyl)-piperazine]-amide is used.

36. Pharmaceutical agents according to claim 1, wherein as fluoroalkyl-containing metal complexes, the compounds with polar radicals of general formula Id 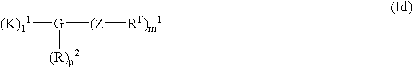
in which
R^{F} is a fluorinated, straight-chain or branched carbon chain with formula —CₙF₂ₙE, in which E represents a terminal fluorine, chlorine, bromine, iodine or hydrogen atom, and n stands for numbers 4-30,
K stands for a metal complex of general formula IId, 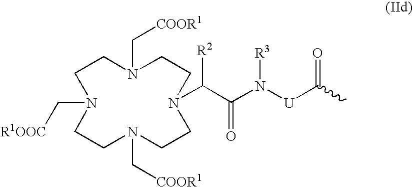
in which
R¹ means a hydrogen atom or a metal ion equivalent of atomic numbers 20-29, 39, 42, 44, 58-70 or 83, provided that at least two R¹ stand for metal ion equivalents,
R² and R³, independently of one another, represent hydrogen, C₁-C₇-alkyl, benzyl, phenyl, —CH₂OH or —CH₂OCH₃, and
U represents —C₆H₄—O—CH₂-ω-, —(CH₂)₁₋₅-ω, a phenylene group, —CH₂—NHCO—CH₂—CH(CH₂COOH)—C₆H₄-ω-, —C₆H₄—(OCH₂CH₂)₀₋₁—N(CH₂COOH)—CH₂-ω or a C₁-C₁₂-alkylene group or C₇-C₁₂-C₆H₄-0 group that optionally is interrupted by one or more oxygen atoms, 1 to 3 —NHCO groups or 1 to 3 —CONH groups and/or substituted with 1 to 3 —(CH₂)₀₋₅COOH groups, whereby ω stands for the binding site to —CO—,
or
of general formula IIId 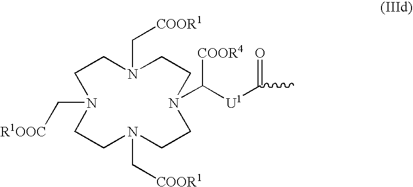
in which R¹ has the above-mentioned meaning, R⁴ represents hydrogen or a metal ion equivalent that is mentioned under R¹, and U¹ represents —C₆H₄—O—CH₂-ω-, whereby ω means the binding site to —CO—
or of general formula IVd 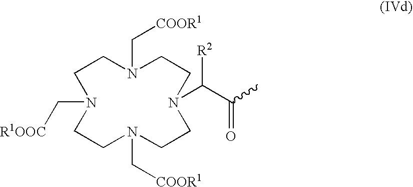
in which R¹ and R² have the above-mentioned meaning,
or of general formula VdA or VdB 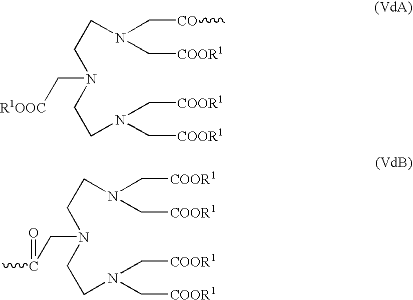
in which R¹ has the above-mentioned meaning,
or of general formula VId 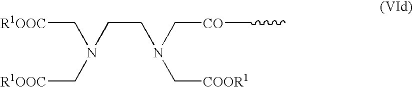
in which R¹ has the above-mentioned meaning,
or of general formula VIId 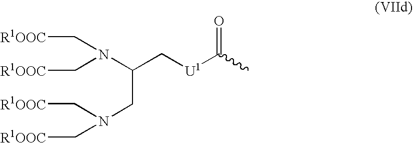
in which R¹ has the above-mentioned meaning, and
U¹ represents —C₆H₄—O—CH₂-ω-, whereby ω means the binding site to —CO—, and in radical K, optionally present free acid groups can optionally be present as salts of organic and/or inorganic bases or amino acids or amino acid amides,
G represents a radical that is functionalized in at least three places and that is selected from the radicals a) to i) below 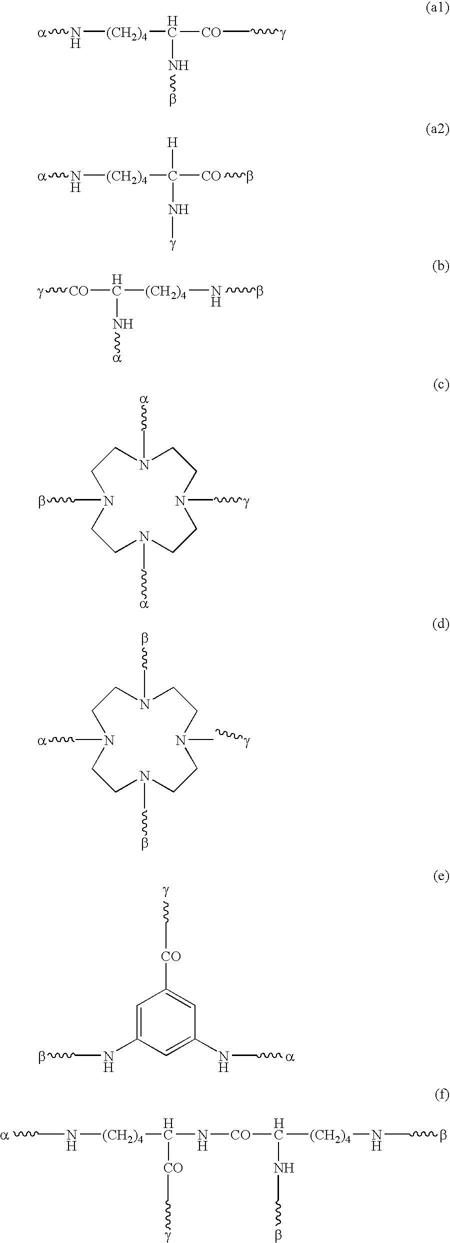 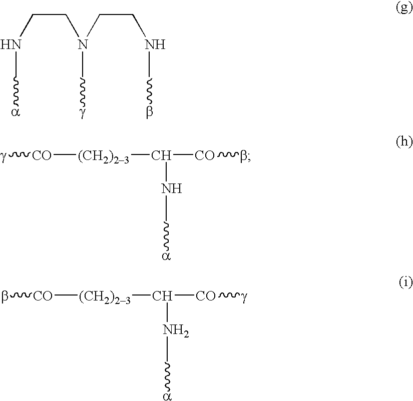
whereby α means the binding site of G to complex K, β is the binding site of G to radical R, and γ represents the binding site of G to radical Z,
Z stands for 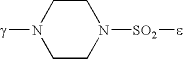
γ-C(O)CH₂O(CH₂)₂-ε,
whereby γ represents the binding site of Z to radical G, and ε means the binding site of Z to fluorinated radical R_{f},
R represents a polar radical that is selected from complexes K of general formulas IId to VId, whereby R¹ here means a hydrogen atom or a metal ion equivalent of atomic numbers 20, 23-29, 42-46 or 58-70, and radicals R², R³, R⁴, U and U¹ have the above-indicated meaning, or
the folic acid radical or
a carbon chain with 2-30 C atoms that is bonded via —CO—, SO₂— or a direct bond to radical G, straight or branched, saturated or unsaturated, optionally interrupted by 1-10 oxygen atoms, 1-5 —NHCO groups, 1-5 —CONH groups, 1-2 sulfur atoms, 1-5 —NH groups, or 1-2 phenylene groups, which optionally can be substituted with 1-2 OH groups, 1-2 NH₂ groups, 1-2 —COOH groups, or 1-2 —SO₃H groups, or
optionally substituted with 1-8 OH groups, 1-5 —COOH groups, 1-2 SO₃H groups, 1-5 NH₂ groups, or 1-5 C₁-C₄-alkoxy groups, and
l¹, m¹, p², independently of one another, mean integer 1 or 2, are used.

37. Pharmaceutical agents according to claim 36, wherein the compounds of general formula Id, in which K stands for a metal complex of general formula IId, IIId, VdB or VId, are used.

38. Pharmaceutical agents according to claim 36, wherein the compounds of general formula Id, in which polar radical R has the meaning of complex K, preferably complexes K of general formula IId, IIId, VdA or VIId, are used.

39. Pharmaceutical agents according to claim 36, wherein the compounds of general formula Id, in which polar radical R has the following meanings:
—C(O)CH₂CH₂SO₃H
—C(O)CH₂OCH₂CH₂OCH₂CH₂OH
—C(O)CH₂OCH₂CH₂OH
—C(O)CH₂OCH₂CH(OH)CH₂OH
—C(O)CH₂NH—C(O)CH₂COOH
—C(O)CH₂CH(OH)CH₂OH
—C(O)CH₂OCH₂COOH
—SO₂CH₂CH₂COOH
—C(O)—C₆H₃-(m-COOH)₂
—C(O)CH₂O(CH₂)₂—C₆H₃-(m-COOH)₂
—C(O)CH₂O—C₆H₄-m-SO₃H
—C(O)CH₂NHC(O)CH₂NHC(O)CH₂OCH₂COOH
—C(O)CH₂OCH₂CH₂OCH₂COOH
—C(O)CH₂OCH₂CH(OH)CH₂O—CH₂CH₂OH
—C(O)CH₂OCH₂CH(OH)CH₂OCH₂—CH(OH)—CH₂OH
—C(O)CH₂SO₃H
—C(O)CH₂CH₂COOH
—C(O)CH(OH)CH(OH)CH₂OH
—C(O)CH₂O[(CH₂)₂O]₁₋₉—CH₃
—C(O)CH₂O[(CH₂)₂O]₁₋₉—H
—C(O)CH₂OCH(CH₂OH)₂
—C(O)CH₂OCH(CH₂OCH₂COOH)₂
—C(O)—C₆H₃-(m-OCH₂COOH)₂
—CO—CH₂O—(CH₂)₂O—(CH₂)₂O—(CH₂)₂O(CH₂)₂OCH₃
preferably —C(O)CH₂O[(CH₂)₂O]₄—CH₃, are used.

40. Pharmaceutical agents according to claim 36, wherein the compounds of general formula Id, in which polar radical R is the folic acid radical, are used.

41. Pharmaceutical agents according to claim 36, wherein the compounds of general formula Id, in which G represents lysine radical (a) or (b), are used.

42. Pharmaceutical agents according to claim 36, wherein the compounds of general formula Id, in which U in metal complex K represents group —CH₂— or —C₆H₄—O—CH₂-ω, whereby ω stands for the binding site to —CO—, are used.

43. Pharmaceutical agents according to claim 36, wherein the gadolinium complex of 2,6-N,N′-bis[1,4,7-tris(carboxylatomethyl)-1,4,7,10-tetraazacyclododecane-10-(pentanoyl-3-aza-4-oxo-5-methyl-5-yl)]-lysine-[1-(4-perfluorooctylsulfonyl-piperazine]-amide is used.

44. Pharmaceutical agents according to claim 1, wherein as fluoroalkyl-containing metal complexes, galenical formulations that contain paramagnetic fluoroalkyl-containing metal complexes of general formula I, Ia, Ib, Ic and/or Id and diamagnetic perfluoroalkyl-containing substances are used.

45. Pharmaceutical agents according to claim 44, wherein as diamagnetic perfluoroalkyl-containing substances, those of general formula XX are used:
R^{F}-L²-B² (XX)
in which R^{F} represents a straight-chain or branched perfluoroalkyl radical with 4 to 30 carbon atoms, L² stands for a linker, and B² stands for a hydrophilic group.

46. Pharmaceutical agents according to claim 45, wherein linker L² is a direct bond, an —SO₂ group, or a straight-chain or branched carbon chain with up to 20 carbon atoms, which can be substituted with one or more —OH, —COO⁻, or —SO₃ groups and/or optionally one or more —O—, —S—, —CO—, —CONH—, —NHCO—, —CONR⁹—, —NR⁹CO—, —SO₂—, —PO₄⁻—, —NH—, or —NR⁹ groups, an aryl ring or a piperazine-diyl radical, whereby R⁹ stands for a C₁- to C₂₀-alkyl radical, which in turn can contain one or more O atoms and/or can be substituted with —COO⁻ or —SO₃ groups.

47. Pharmaceutical agents according to claim 45, wherein hydrophilic group B² is a mono- or disaccharide radical, which optionally is substituted with one or more adjacent —COO⁻ or —SO₃⁻ groups; a dicarboxylic acid radical, an isophthalic acid radical, a picolinic acid radical, a benzenesulfonic acid radical, a tetrahydropyrandicarboxylic acid radical, a 2,6-pyridine-dicarboxylic acid radical, a quaternary ammonium ion radical, an aminopolycarboxylic acid radical, an aminodipolyethylene glycolsulfonic acid radical, an aminopolyethylene glycol group, an —SO₂—(CH₂)₂—OH group, a polyhydroxyalkyl chain with at least two hydroxyl groups or one or more polyethylene glycol chains with at least two glycol units, whereby the polyethylene glycol chains are terminated by an —OH or —OCH₃ group.

48. Pharmaceutical agents according to claim 44, wherein as diamagnetic perfluoroalkyl-containing substances, conjugates that consist of α-, β-, or γ-cyclodextrin and compounds of general formula XXI are used:
A²-L³-R^{F} (XXI)
in which A² stands for an adamantane, biphenyl or anthracene molecule, L³ stands for a linker, and R^{F} stands for a straight-chain or branched perfluoroalkyl radical with 4 to 30 carbon atoms, and whereby linker L³ is a straight-chain hydrocarbon chain with 1 to 20 carbon atoms, which can be interrupted by one or more oxygen atoms, one or more —CO—, —SO₂—, —CONH', —NHCO—, CONR¹⁰—, —NR¹⁰CO—, —NH—, or —N(R¹⁰)— groups or a piperazine-diyl radical, whereby R¹⁰ is a C₁-C₅-alkyl radical.

49. Pharmaceutical agents according to claim 44, wherein as diamagnetic perfluoroalkyl-containing substances, those of general formula XXII are used:
R^{F}—X¹ (XXII)
in which R^{F} represents a straight-chain or branched perfluoroalkyl radical with 4 to 30 carbon atoms, and X¹ is a radical that is selected from the group of the following radicals (in this case, n is a number between 1 and 10):

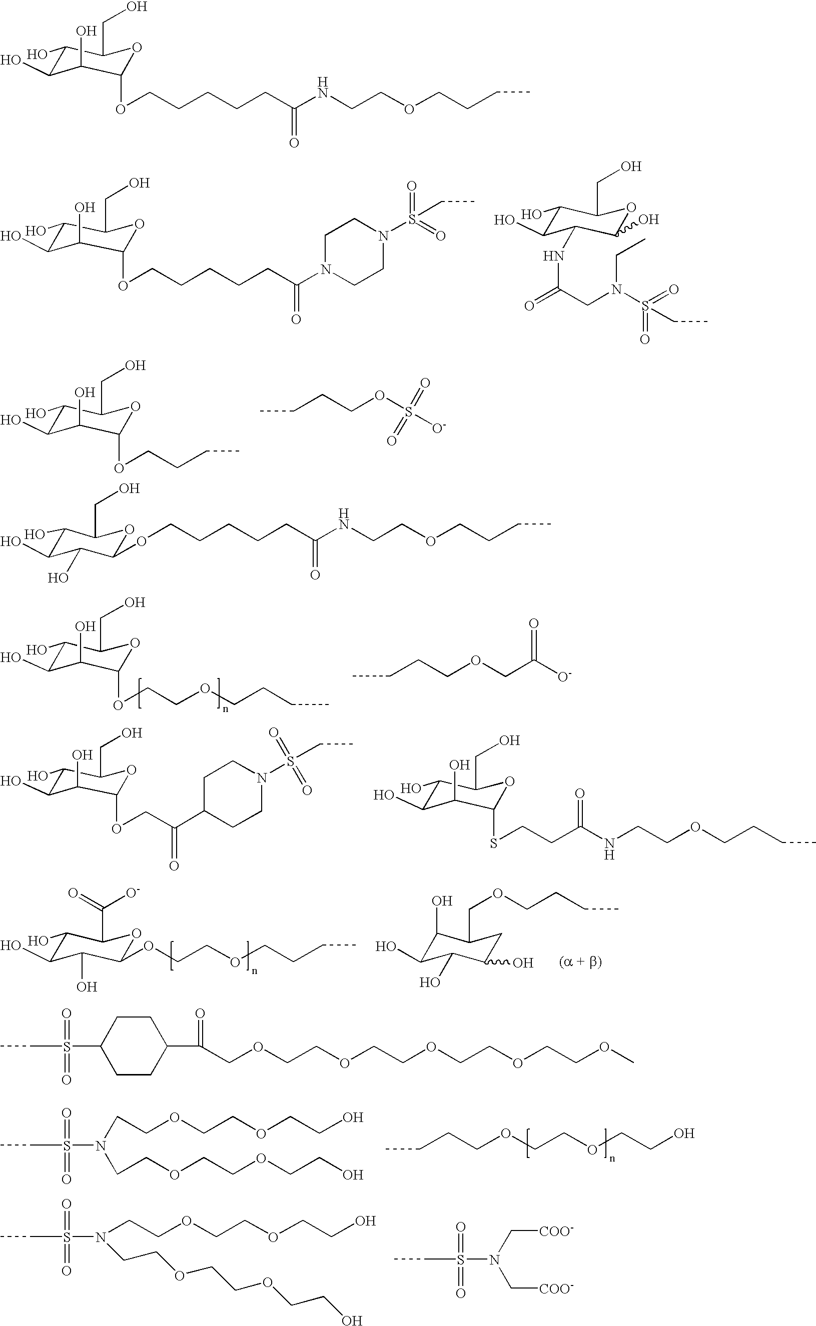

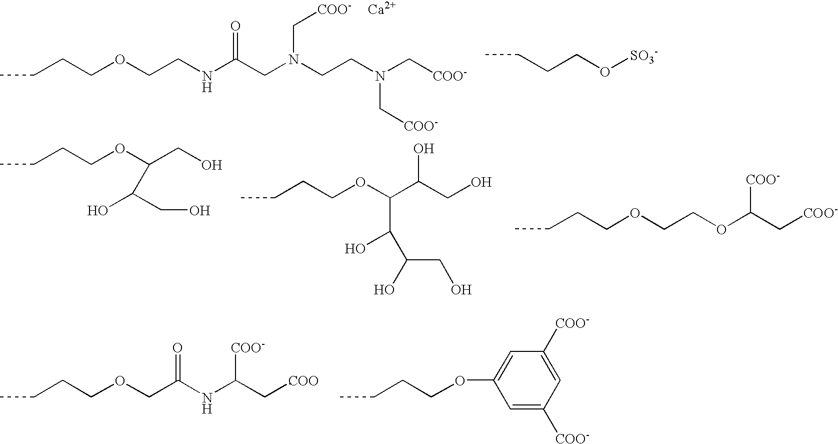

50. Pharmaceutical agents according to claim 1, wherein they contain at least one of the following epothilones:
(4S,7R,8S,9S,13Z,16S(E))-4,8-Dihydroxy-5,5,7,9,13-pentamethyl-16-[1-methyl-2-(2-methyl-thiazol-4-yl)-vinyl]-oxacyclohexadec-13-ene-2,6-dione
(4S,7R,8S,9S,13Z,16S(E))-4,8-Dihydroxy-16-[2-(2-hydroxymethyl-thiazol-4-yl)-1-methyl-vinyl]-5,5,7,9,13-pentamethyl-oxacyclohexadec-13-ene-2,6-dione
(4S,7R,8S,9S,13Z,16S(E))-16-[2-(2-Aminomethyl-thiazol-4-yl)-1-methyl-vinyl[-4,8-dihydroxy-5,5,7,9,13-pentamethyl-oxacyclohexadec-13-ene-2,6-dione
(1S,3S(E),7S,10R,11S,12S,16R)-7,11-Dihydroxy-8,8,10,12,16-pentamethyl-3-[1-methyl-2-(2-methyl-thiazol-4-yl)-vinyl]-4,17-dioxa-bicyclo[14.1.0]heptadecane-5,9-dione
(1S,3S(E),7S,10R,11S,12S,16R)-7,11-Dihydroxy-3-[2-(2-hydroxymethyl-thiazol-4-yl)-1-methyl-vinyl]-8,8,10,12,16-pentamethyl-4,17-dioxa-bicyclo[14.1.0]heptadecane-5,9-dione
(1S,3S(E),7S,10R,11S,12S,16R)-3-[2-(2-Aminomethyl-thiazol-4-yl)-1-methyl-vinyl]-7,11-dihydroxy-8,8,10,12,16-pentamethyl-4,17-dioxa-bicyclo[14.1.0]heptadecane-5,9-dione
(4S,7R,8S,9S,13Z,16S(E))-4,8-Dihydroxy-7-ethyl-5,5,9,13-tetramethyl-16-[1-methyl-2-(2-methyl-thiazol-4-yl)-vinyl]-oxacyclohexadec-13-ene-2,6-dione
(4S,7R,8S,9S,13Z,16S(E))-4,8-Dihydroxy-16-[2-(2hydroxymethyl-thiazol-4-yl)-1-methyl-vinyl]-7-ethyl-5,5,9,13-tetramethyl-oxacyclohexadec-13-ene-2,6-dione
(4S,7R,8S,9S,13Z,16S(E))-16-[2-(2-Aminomethyl-thiazol-4-yl)-1-methyl-vinyl]4,8-dihydroxy-7-ethyl-5,5,9,13-tetramethyl-oxacyclohexadec-13-ene-2,6-dione
(1S,3S(E),7S,10R,11S,12S,16R)-7,11-Dihydroxy-10-ethyl-8,8,12,16-tetramethyl-3-[1-methyl-2-(2-methyl-thiazol-4-yl)-vinyl]-4,17-dioxa-bicyclo[14.1.0]heptadecane-5,9-dione
(1S,3S(E),7S,10R,11S,12S,16R)-7,11-Dihydroxy-3-[2-(2-hydroxymethyl-thiazol-4-yl)-1-methyl-vinyl]-10-ethyl-8,8,12,16-tetramethyl-4,17-dioxa-bicyclo[14.1.0]-heptadecane-5,9-dione
(1S,3S(E),7S,10R,11S,12S,16R)-3-[2-(2-Aminomethyl-thiazol-4-yl)-1-methyl-vinyl]-7,11-dihydroxy-10-ethyl-8,8,12,16-tetramethyl-4,17-dioxa-bicyclo[14.1.0]-heptadecane-5,9-dione
(4S,7R,8S,9S,13Z,16S(Z))-4,8-Dihydroxy-5,5,7,9,13-pentamethyl-16-[1-fluoro-2-(2-methyl-thiazol-4-yl)-vinyl]-oxacyclohexadec-13-ene-2,6-dione
(4S,7R,8S,9S,13Z,16S(Z))-4,8-Dihydroxy-16-[2-(2-hydroxymethyl-thiazol-4-yl)-1-fluoro-vinyl]-5,5,7,9,13-pentamethyl-oxacyclohexadec-13-ene-2,6-dione
(4S,7R,8S,9S,13Z,16S(Z))-16-[2-(2-Aminomethyl-thiazol-4-yl)-1-fluoro-vinyl]-4,8-dihydroxy-5,5,7,9,13-pentamethyl-oxacyclohexadec-13-ene-2,6-dione
(1S,3S(Z),7S,10R,11S,12S,16R)-7,11-Dihydroxy-8,8,10,12,16-pentamethyl-3-[1-fluoro-2-(2-methyl-thiazol-4-yl)-vinyl]-4,17-dioxa-bicyclo[14.1.0]heptadecane-5,9-dione
(1S,3S(Z),7S,10R,11S,12S,16R)-7,11-Dihydroxy-3-[2-(2-hydroxymethyl-thiazol-4-yl)-1-fluoro-vinyl]-8,8,10,12,16-pentamethyl-4,17-dioxa-bicyclo[14.1.0]heptadecane-5,9-dione
(1S,3S(Z),7S,10R,11S,12S,16R-3-[2-(2-Aminomethyl-thiazol-4-yl)-1-fluoro-vinyl]-7,11-dihydroxy-8,8,10,12,16-pentamethyl-4,17-dioxa-bicyclo[14.1.0]heptadecane-5,9-dione
(4S,7R,8S,9S,13Z,16S(Z))-4,8-Dihydroxy-5,5,7,9,13-pentamethyl-16-[1-chloro-2-(2-methyl-thiazol-4-yl)-vinyl]-oxacyclohexadec-13-ene-2,6-dione
(4S,7R,8S,9S,13Z,16S(Z))-4,8-Dihydroxy-16-[2-(2-hydroxymethyl-thiazol-4-yl)-1-chloro-vinyl]-5,5,7,9,13-pentamethyl-oxacyclohexadec-13-ene-2,6-dione
(4S,7R,8S,9S,13Z,16S(Z))-16-[2-(2-Aminomethyl-thiazol-4-yl)-1-chloro-vinyl]-4,8-dihydroxy-5,5,7,9,13-pentamethyl-oxacyclohexadec-13-ene-2,6-dione
(1S,3S(Z),7S,10R,11S,12S,16R)-7,11-Dihydroxy-8,8,10,12,16-pentamethyl-3-[1-chloro-2-(2-methyl-thiazol-4-yl)-vinyl]-4,17-dioxa-bicyclo[14.1.0]heptadecane-5,9-dione
(1S,3S(Z),7S,10R,11S,12S,16R)-7,11-Dihydroxy-3-[2-(2-hydroxymethyl-thiazol-4-yl)-1-chloro-vinyl]-8,8,10,12,16-pentamethyl-4,17-dioxa-bicyclo[14.1.0]heptadecane-5,9-dione
(1S,3S(Z),7S,10R,11S,12S,16R)-3-[2-(2-Aminomethyl-thiazol-4-yl)-1-chloro-vinyl]-7,11-dihydroxy-8,8,10,12,16-pentamethyl-4,17-dioxa-bicyclo[14.1.0]heptadecane-5,9-dione
(4S,7R,8S,9S,13Z,16S(Z))-4,8-Dihydroxy-7-ethyl-5,5,9,13-tetramethyl-16-[1-fluoror-2-(2-methyl-thiazol-4-yl)-vinyl]-oxacyclohexadec-13-ene-2,6-dione
(4S,7R,8S,9S,13Z,16S(Z))-4,8-Dihydroxy-16-[2-(2-hydroxymethyl-thiazol-4-yl)-1-fluoro-vinyl]-7-ethyl-5,5,9,13-tetramethyl-oxacyclohexadec-13-ene-2,6-dione
(4S,7R,8S ,9S,13Z,16S(Z))-16-[2-(2-Aminomethyl-thiazol-4-yl)-1-fluoro-vinyl]-4,8-dihydroxy-7-ethyl-5,5,9,13-tetramethyl-oxacyclohexadec-13-ene-2,6-dione
(1S,3S(Z),7S,10R,11S,12S,16R)-7,11-Dihydroxy-10-ethyl-8,8,12,16-tetramethyl-3-[1-fluoro-2-(2-methyl-thiazol-4-yl)-vinyl]-4,17-dioxa-bicyclo[14.1.0]heptadecane-5,9-dione
(1S,3S(Z),7S,10R,11S,12S,16R)-7,11-Dihydroxy-3-[2-(2-hydroxymethyl-thiazol-4-yl)-1fluoro-vinyl]-10-ethyl-8,8,12,16-tetramethyl-4,17-dioxa-bicyclo[14.1.0]-heptadecane-5,9-dione
(1S,3S(Z),7S,10R,11S,12S,16R-3-[2-(2-Aminomethyl-thiazol-4-yl)-1-fluoro-vinyl]-7,11-dihydroxy-10-ethyl-8,8,12,16-tetramethyl14,17-dioxa-bicyclo[14.1.0]-heptadecane-5,9-dione
(4S,7R,8S,9S,13Z,16S(Z))-4,8-Dihydroxy-7-ethyl-5,5,9,13-tetramethyl-16-[1-chloro-2-(2-methyl-thiazol4-yl)-vinyl]-oxacyclohexadec-13-ene-2,6-dione
(4S,7R,8S,9S,13Z,16S(Z))-4,8-Dihydroxy-16-[2-(2-hydroxymethyl-thiazol-4-yl)-1-chloro-vinyl]-7-ethyl-5,5,9,13-tetramethyl-oxacyclohexadec-13-ene-2,6-dione
(4S,7R,8S,9S,13Z,16S(Z))-16-[2-(2-Aminomethyl-thiazol-4-yl)-1-chloro-vinyl]-4,8-dihydroxy-7-ethyl-5,5,9,13-tetramethyl-oxacyclohexadec-13-ene-2,6-dione
(1S,3S(Z),7S,10R,11S,12S,16R)-7,11-Dihydroxy-10-ethyl-8,8,12,16-tetramethyl-3-[1-chloro-2-(2-methyl-thiazol-4-yl)-vinyl]-4,17-dioxa-bicyclo[14.1.0]heptadecane-5,9-dione
(1S,3S(Z),7S,10R,11S,12S,16R)-7,11-Dihydroxy-3-[2-(2-hydroxymethyl-thiazol-4-yl)-1-chloro-vinyl]-10-ethyl-8,8,12,16-tetramethyl-4,17-dioxa-bicyclo[14.1.0]-heptadecane-5,9-dione
(1S,3S(Z),7S,10R,11S,12S,16R-3-[2-(2-Aminomethyl-thiazol-4-yl)-1-chloro-vinyl]-7,11-dihydroxy-10-ethyl-8,8,12,16-tetramethyl-4,17-dioxa-bicyclo[14.1.0]-heptadecane-5,9-dione
(4S,7R,8S,9S,13Z,16S(E)-4,8-Dihydroxy-5,5,7,9,13-pentamethyl-16-[1-methyl-2-(2-pyridyl)-vinyl]-oxacyclohexadec-13-ene-2,6-dione
(1S,3S(E),7S,10R,11S,12S,16R)-7,11-Dihydroxy-8,8,10,12,16-pentamethyl-3-[1-methyl-2-(2-pyridyl)-vinyl]-4,17-dioxa-bicyclo[14.1.0]heptadecane-5,9-dione
(4S,7R,8S,9S,13Z,16S(E)-4,8-Dihydroxy-7-ethyl-5,5,9,13-tetramethyl-16-[1-methyl-2-(2-pyridyl)-vinyl]-oxacyclohexadec-13-ene-2,6-dione
(1S,3S(E),7S,10R,11S,12S,16R)-7,11-Dihydroxy-10-ethyl-8,8,12,16-tetramethyl-3-[1-methyl-2-(2-pyridyl)-vinyl]-4,17-dioxa-bicyclo[14.1.0]heptadecane-5,9-dione
(4S,7R,8S,9S,13Z,16S(Z))-4,8-Dihydroxy-5,5,7,9,13-pentamethyl-16-[1-fluoro-2-(2-pyridyl)-vinyl]-oxacyclohexadec-13-ene-2,6-dione
(1S,3S(Z),7S,10R,11S,12S,16R)-7,11-Dihydroxy-8,8,10,12,16-pentamethyl-3-[1-fluoro-2-(2-pyridyl)-vinyl]-4,17-dioxa-bicyclo[14.1.0]heptadecane-5,9-dione
(4S,7R,8S,9S,13Z,16S(Z))-4,8-Dihydroxy-5,5,7,9,13-pentamethyl-16-[1-chloro-2-(2-pyridyl)-vinyl]-oxacyclohexadec-13-ene-2,6-dione
(1S,3S(Z),7S,10R,11S,12S,16R)-7,11-Dihydroxy-8,8,10,12,16-pentamethyl-3-[1-chloro-2-(2-pyridyl)-vinyl]-4,17-dioxa-bicyclo[14.1.0]heptadecane-5,9-dione
(4S,7R,8S,9S,13Z,16S(Z))-4,8-Dihydroxy-7-ethyl-5,5,9,13-tetramethyl-16-[1-fluoro-2-(2-pyridyl)-vinyl]-oxacyclohexadec-13-ene-2,6-dione
(1S,3S(Z),7S,10R,11S,12S,16R)-7,11-Dihydroxy-10-ethyl-8,8,12,16-tetramethyl-3-[1-fluoro-2-(2-pyridyl)-vinyl]-4,17-dioxa-bicyclo[14.1.0]heptadecane-5,9-dione
(4S,7R,8S,9S,13Z,16S(Z))-4,8-Dihydroxy-7-ethyl-5,5,9,13-tetramethyl-16-[1-chloror-2-(2-pyridyl)-vinyl]-oxacyclohexadec-13-ene-2,6-dione
(1S,3S(Z),7S,10R,11S,12S,16R)-7,11-Dihydroxy-10-ethyl-8,8,12,16-tetramethyl-3-[1-chloro-2-(2-pyridyl)-vinyl]-4,17-dioxa-bicyclo[14.1.0]heptadecane-5,9-dione
(4S,7R,8S,9S,13Z,16S(E)-4,8-Dihydroxy-5,5,7,9,13-pentamethyl-16-[1-methyl-2-(2-methyl-oxazol-4-yl)-vinyl]-oxacyclohexadec-13-ene-2,6-dione
(4S,7R,8S,9S,13Z,16S(E))-4,8-Dihydroxy-16-[2-(2-hydroxymethyl-oxazol-4-yl)-1-methyl-vinyl]-5,5,7,9,13-pentamethyl-oxacyclohexadec-13-ene-2,6-dione
(4S,7R,8S,9S,13Z,16S(E))-16-[2-(2-Aminomethyl-oxazol-4-yl)-1-methyl-vinyl[-4,8-dihydroxy-5,5,7,9,13-pentamethyl-oxacyclohexadec-13-ene-2,6-dione
(1S,3S(E),7S,10R,11S,12S,16R)-7,11-Dihydroxy-8,8,10,12,16-pentamethyl-3-[1-methyl-2-(2-methyl-oxazol4-yl)-vinyl]-4,17-dioxa-bicyclo[14.1.0]heptadecane-5,9-dione
(1S,3S(E),7S,10R,11S,12S,16R)-7,11-Dihydroxy-3-[2-(2-hydroxymethyl-oxazol-4-yl)-1methyl-vinyl]-8,8,10,12,16-pentamethyl-4,17-dioxa-bicyclo[14.10.]heptadecane-5,9-dione
(1S,3S(E),7S,10R,11S,12S,16R)-3-[2-(2-Aminomethyl-oxazol-4-yl)-1-methyl-vinyl]-7,11-dihydroxy-8,8,10,12,16-pentamethyl-4,17-dioxa-bicyclo[14.10.]heptadecane-5,9-dione
(4S,7R,8S,9S,13Z,16S(E)-4,8-Dihydroxy-7-ethyl-5,5,9,13-tetramethyl-16-[1-methyl-2-(2-methyl-oxazol-4-yl)-vinyl]-oxacyclohexadec-13-ene-2,6-dione
(4S,7R,8S,9S,13Z,16S(E))-4,8-Dihydroxy-16-[2-(2-hydroxymethyl-oxazol-4-yl)-1-methyl-vinyl]-7-ethyl-5,5,9,13-tetramethyl-oxacyclohexadec-13-ene-2,6-dione
(4S,7R,8S,9S,13Z,16S(E))-16-[2-(2-Aminomethyl-oxazol-4-yl)-1-methyl-vinyl]-4,8-dihydroxy-7-ethyl-5,5,9,13-tetramethyl-oxacyclohexadec-13-ene-2,6-dione
(1S,3S(E),7S,10R,11S,12S,16R)-7,11-Dihydroxy-10-ethyl-8,8,12,16-tetramethyl-3-[1-methyl-2-(2-methyl-oxazol-4-yl)-vinyl]-4,17-dioxa-bicyclo[14.1.0]heptadecane-5,9-dione
(1S,3S(E),7S,10R,11S,12S,16R)-7,11-Dihydroxy-3-[2-(2-hydroxymethyl-oxazol-4-yl)-1-methyl-vinyl]-10-ethyl-8,8,12,16-tetramethyl-4,17-dioxa-bicyclo[14.1.0]-heptadecane-5,9-dione
(1S,3S(E),7S,10R,11S,12S,16R)-3-[2-(2-Aminomethyl-oxazol-4-yl)-1-methyl-vinyl]-7,11-dihydroxy-10-ethyl-8,8,12,16-tetramethyl-4,17-dioxa-bicyclo[14.1.0]-heptadecane-5,9-dione
(4S,7R,8S,9S,13Z,16S(Z))-4,8-Dihydroxy-5,5,7,9,13-pentamethyl-16-[1-fluoro-2-(2-methyl-oxazol-4-yl)-vinyl]-oxacyclohexadec-13-ene-2,6-dione
(4S,7R,8S,9S,13Z,16S(Z))-4,8-Dihydroxy-16-[2-(2-hydroxymethyl-oxazol-4-yl)-1-fluoro-vinyl]-5,5,7,9,13-pentamethyl-oxacyclohexadec-13-ene-2,6-dione
(4S,7R,8S,9S,13Z,16S(Z))-16-[2-(2-Aminomethyl-oxazol-4-yl)-1-fluoro-vinyl]-4,8-dihydroxy-5,5,7,9,13-pentamethyl-oxacyclohexadec-13-ene-2,6-dione
(1S,3S(Z),7S,10R,11S,12S,16R)-7,11-Dihydroxy-8,8,10,12,16-pentamethyl-3-[1-fluoro-2-(2-methyl-oxazol-4-yl)-vinyl]-4,17-dioxa-bicyclo[14.1.0]heptadecane-5,9-dione
(1S,3S(Z),7S,10R,11S,12S,16R)-7,11-Dihydroxy-3-[2-(2-hydroxymethyl-oxazol-4-yl)-1-fluoro-vinyl]-8,8,10,12,16-pentamethyl-4,17-dioxa-bicyclo[14.1.0]heptadecane-5,9-dione
(1S,3S(Z),7S,10R,11S,12S,16R)-3-[2-(2-Aminomethyl-oxazol-4-yl)-1-fluoro-vinyl]-7,11-dihydroxy-8,8,10,12,16-pentamethyl-4,17-dioxa-bicyclo[14.1.]heptadecane-5,9-dione
(4S,7R,8S,9S,13Z,16S(Z))-4,8-Dihydroxy-5,5,7,9,13-pentamethyl-16-[1-chloro-2-(2-methyl-oxazol-4-yl)-vinyl]-oxacyclohexadec-13-ene-2,6-dione
(4S,7R,8S,9S,13Z,16S(Z))-4,8-Dihydroxy-16-[2-(2-hydroxymethyl-oxazol-4-yl)-1-chloro-vinyl]-5,5,7,9,13-pentamethyl-oxacyclohexadec-13-ene-2,6-dione
(4S,7R,8S,9S,13Z,16S(Z))-16-[2-(2-Aminomethyl-oxazol-4-yl)-1-chloro-vinyl]-4,8-dihydroxy-5,5,7,9,13-pentamethyl-oxacyclohexadec-13-ene-2,6-dione
(1S,3S(Z),7S,10R,11S,12S,16R)-7,11-Dihydroxy-8,8,10,12,16-pentamethyl-3-[1-chloro-2-(2-methyl-oxazol-4-yl)-vinyl]-4,17-dioxa-bicyclo[14.1.0]heptadecane-5,9-dione
(1S,3S(Z),7S,10R,11S,12S,16R)-7,11-Dihydroxy-3-[2-(2-hydroxymethyl-oxazol-4-yl)-1-chloro-vinyl]-8,8,10,12,16-pentamethyl-4,17-dioxa-bicyclo[14.1.0]heptadecane-5,9-dione
(1S,3S(Z),7S,10R,11S,12S,16R)-3-[2-(2-Aminomethyl-oxazol-4-yl)-1-chloro-vinyl]-7,11-dihydroxy-8,8,10,12,16-pentamethyl-4,17-dioxa-bicyclo[14.1.0]heptadecane-5,9-dione
(4S,7R,8S,9S,13Z,16S(Z))-4,8-Dihydroxy-7-ethyl-5,5,9,13-tetramethyl-16-[1-fluoro-2-(2-methyl-oxazol-4-yl)-vinyl]-oxacyclohexadec-13-ene-2,6-dione
(4S,7R,8S,9S,13Z,16S(Z))-4,8-Dihydroxy-16-[2-(2-hydroxymethyl-oxazol-4-yl)-1-fluoro-vinyl]-7-ethyl-5,5,9,13-tetramethyl-oxacyclohexadec-13-ene-2,6-dione
(4S,7R,8S,9S,13Z,16S(Z))-16-[2-(2-Aminomethyl-oxazol-4-yl)-1-fluoro-vinyl]-4,8-dihydroxy-7-ethyl-5,5,9,13-tetramethyl-oxacyclohexadec-13-ene-2,6-dione
(1S,3S(Z),7S,10R,11S,12S,16R)-7,11-Dihydroxy-10-ethyl-8,8,12,16-tetramethyl-3-[1-fluoro-2-(2-methyl-oxazol-4-yl)-vinyl]-4,17-dioxa-bicyclo[14.1.0]heptadecane-5,9-dione
(1S,3S(Z),7S,10R,11S,12S,16R)-7,11-Dihydroxy-3-[2-(2-hydroxymethyl-oxazol-4-yl)-1-fluoro-vinyl]-10-ethyl-8,8,12,16-tetramethyl-4,17-dioxa-bicyclo[14.1.0]-heptadecane-5,9-dione
(1S,3S(Z),7S,10R,11S,12S,16R)-3-[2-(2-Aminomethyl-oxazol-4-yl)-1-fluoro-vinyl]-7,11-dihydroxy-10-ethyl-8,8,12,16-tetramethyl-4,17-dioxa-bicyclo[14.1.0]-heptadecane-5,9-dione
(4S,7R,8S,9S,13Z,16S(Z))-4,8-Dihydroxy-7-ethyl-5,5,9,13-tetramethyl-16-[1-chloro-2-(2-methyl-oxazol-4-yl)-vinyl]-oxacyclohexadec-13-ene-2,6-dione
(4S,7R,8S,9S,13Z,16S(Z))-4,8-Dihydroxy-16-[2-(2-hydroxymethyl-oxazol-4-yl)-1-chloro-vinyl]-7-ethyl-5,5,9,13-tetramethyl-oxacyclohexadec-13-ene-2,6-dione
(4S,7R,8S,9S,13Z,16S(Z))-16-[2-(2-Aminomethyl-oxazol-4-yl)-1-chloro-vinyl]-4,8-dihydroxy-7-ethyl-5,5,9,13-tetramethyl-oxacyclohexadec-13-ene-2,6-dione
(1S,3S(Z),7S,10R,11S,12S,16R)-7,11-Dihydroxy-10-ethyl-8,8,12,16-tetramethyl-3-[1-chloro-2-(2-methyl-oxazol-4-yl)-vinyl]-4,17-dioxa-bicyclo[14.1.0]heptadecane-5,9-dione
(1S,3S(Z),7S,10R,11S,12S,16R)-7,11-Dihydroxy-3-[2-(2-hydroxymethyl-oxazol-4-yl)-1-chloro-vinyl]-10-ethyl-8,8,12,16-tetramethyl-4,17-dioxa-bicyclo[14.1.0]-heptadecane-5,9-dione
(1S,3S(Z),7S,10R,11S,12S,16R)-3-[2-(2-Aminomethyl-oxazol-4-yl)-1-chloro-vinyl]-7,11-dihydroxy-10-ethyl-8,8,12,16-tetramethyl-4,17-dioxa-bicyclo[14.1.0]-heptadecane-5,9-dione
(4S,7R,8S,9S,13Z,16S(E))-4,8-Dihydroxy-5,5,7,9,13-pentamethyl-16-[2-(2-methyl-thiazol-4-yl)-vinyl]-oxacyclohexadec-13-ene-2,6-dione
(4S,7R,8S,9S,13Z,16S(E))-4,8-Dihydroxy-16-[2-(2-hydroxymethyl-thiazol-4-yl)-vinyl]-5,5,7,9,13-pentamethyl-oxacyclohexadec-13-ene-2,6-dione
(4S,7R,8S,9S,13Z,16S(E))-16-[2-(2-Aminomethyl-thiazol-4-yl)-vinyl]-4,8-dihydroxy-5,5,7,9,13-pentamethyl-oxacyclohexadec-13-ene-2,6-dione
(1S,3S(E),7S,10R,11S,12S,16R)-7,11-Dihydroxy-8,8,10,12,16-pentamethyl-3-[2-(2-methyl-thiazol-4-yl)-vinyl]-4,17-dioxa-bicyclo[14.1.0]heptadecane-5,9-dione
(1S,3S(E),7S,10R,11S,12S,16R)-7,11-Dihydroxy-3-[2-(2-hydroxymethyl-thiazol-4-yl)-vinyl]-8,8,10,12,16-pentamethyl-4,17-dioxa-bicyclo[14.1.0]heptadecane-5,9-dione
(1S,3S(E),7S,10R,11S,12S,16R)-3-[2-(2-Aminomethyl-thiazol-4-yl)-vinyl]-7,11-dihydroxy-8,8,10,12,16-pentamethyl-4,17-dioxa-bicyclo[14.1.0]heptadecane-5,9-dione
(4S,7R,8S,9S,13Z,16S(E))-4,8-Dihydroxy-7-ethyl-5,5,9,13-tetramethyl-16-[2-(2-methyl-thiazol-4-yl)-vinyl]-oxacyclohexadec-13-ene-2,6-dione
(4S,7R,8S,9S,13Z,16S(E))-4,8-Dihydroxy-16-[2-(2-hydroxymethyl-thiazol-4-yl)-vinyl]-7-ethyl-5,5,9,13-tetramethyl-oxacyclohexadec-13-ene-2,6-dione
(4S,7R,8S,9S,13Z,16S(E))-16-[2-(2-Aminomethyl-thiazol-4-yl)-vinyl]-4,8-dihydroxy-7-ethyl-5,5,9,13-tetramethyl-oxacyclohexadec-13-ene-2,6-dione
(1S,3S(E),7S,10R,11S,12S,16R)-7,11-Dihydroxy-10-ethyl-8,8,12,16-tetramethyl-3-[2-(2-methyl-thiazol-4-yl)-vinyl]-4,17-dioxa-bicyclo[14.1.0]heptadecane-5,9-dione
(1S,3S(E),7S,10R,11S,12S,16R)-7,11-Dihydroxy-3-[2-(2-hydroxymethyl-thiazol-4-yl)-vinyl]-10-ethyl-8,8,12,16-tetramethyl-4,17-dioxa-bicyclo[14.1.0]heptadecane-5,9-dione
(1S,3S(E),7S,10R,11S,12S,16R)-3-[2-(2-Aminomethyl-thiazol-4-yl)-vinyl]-7,11-dihydroxy-10-ethyl-8,8,12,16-tetramethyl-4,17-dioxa-bicyclo[14.1.0]heptadecane-5,9-dione
(4S,7R,8S,9S,13Z,16S(E))-4,8-Dihydroxy-5,5,7,9,13-pentamethyl-16-[2-(2-pyridyl)-vinyl]-oxacyclohexadec-13-ene-2,6-dione
(1S,3S(E),7S,10R,11S,12S,16R)-7,11-Dihydroxy-8,8,10,12,16-pentamethyl-3-[2-(2-pyridyl)-vinyl]-4,17-dioxa-bicyclo[14.1.0]heptadecane-5,9-dione
(4S,7R,8S,9S,13Z,16S(E))-4,8-Dihydroxy-7-ethyl-5,5,9,13-tetramethyl-16-[2-pyridyl)-vinyl]-oxacyclohexadec-13-ene-2,6-dione
(1S,3S(E),7S,10R,11S,12S,16R)-7,11-Dihydroxy-10-ethyl-8,8,12,16-tetramethyl-3-[2-(2-pyridyl)-vinyl]-4,17-dioxa-bicyclo[14.1.0]heptadecane-5,9-dione
(4S,7R,8S,9S,13Z,16S)-4,8-Dihydroxy-5,5,7,9,13-pentamethyl-16-(2-methyl-benzothiazol-5-yl)-oxacyclohexadec-13-ene-2,6-dione
(4S,7R,8S,9S,13Z,16S)-4,8-Dihydroxy-16-(2-hydroxymethyl-benzothiazol-5-yl)-5,5,7,9,13-pentamethyl-oxacyclohexadec-13-ene-2,6-dione
(4S,7R,8S,9S,13Z,16S)-16-(2-Aminomethyl-benzothiazol-5-yl)-4,8-dihydroxy-5,5,7,9,13-pentamethyl-oxacyclohexadec-13-ene-2,6-dione
(1S,3S,7S,10R,11S,12S,16R)-7,11-Dihydroxy-8,8,10,12,16-pentamethyl-3-(2-methyl-benzothiazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzothiazol-5-yl)-8,8,10,12,16-pentamethyl-4,17-dioxa-bicyclo[14.1.0]heptadecane-5,9-dione
(1S,3S,7S,10R,11S,12S,16R)-3-(2-Aminomethyl-benzothiazol-5-yl)-7,11-dihydroxy-8,8,10,12,16-pentamethyl-4,17-dioxa-bicyclo[14.1.0]heptadecane-5,9-dione
(4S,7R,8S,9S,13Z,16S)-4,8-Dihydroxy-7-ethyl-5,5,9,13-tetramethyl-16-(2-methyl-benzothiazol-5-yl)oxacyclohexadec-13-ene-2,6-dione
(4S,7R,8S,9S,13Z,16S)-4,8-Dihydroxy-16-(2-hydroxymethyl-benzothiazol-5-yl)-7-ethyl-5,5,9,13-tetramethyl-oxacyclohexadec-13-ene-2,6-dione
(4S,7R,8S,9S,13Z,16S)-16-(2-Aminomethyl-benzothiazol-5-yl)-4,8-dihydroxy-7-ethyl-5,5,9,13-tetramethyl-oxacyclohexadec-13-ene-2,6-dione
(1S,3S,7S,10R,11S,12S,16R)-7,11-Dihydroxy-10-ethyl-8,8,12,16-tetramethyl-3-(2-methyl-benzothiazol-5-y)-4,17-dioxa-bicyclo[14.1.0]heptadecane-5,9-dione
(1S,3S,7S,10R,11S,12S,16R)-7,11-Dihydroxy-3-(2-hydroxymethyl-benzothiazol-5-yl)-10-ethyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzothiazol-5-yl)-7,11-dihydroxy-10-ethyl-8,8,12,16-tetramethyl-4,17-dioxa-bicyclo[14.1.0]heptadecane-5,9-dione
(4S,7R,8S,9S,13Z,16S)-4,8-Dihydroxy-7-propyl-5,5,9,13-tetramethyl-16-(2-methyl-benzothiazol-5-yl)-oxacyclohexadec-13-ene-2,6-dione
(4S,7R,8S,9S,13Z,16S)-4,8-Dihydroxy-16-(2-hydroxymethyl-benzothiazol-5-yl)-7-propyl-5,5,9,13-tetramethyl-oxacyclohexadec-13-ene-2,6-dione
(4S,7R,8S,9S,13Z,16S)-16-(2-Aminomethyl-benzothiazol-5-yl)-4,8-dihydroxy-7-propyl-5,5,9,13-tetramethyl-oxacyclohexadec-13-ene-2,6-dione
(1S,3S,7S,10R,11S,12S,16R)-7,11-Dihydroxy-10-propyl-8,8,12,16-tetramethyl-3-(2-methyl-benzothiazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzothiazol-5-yl)-10-propyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzothiazol-5-yl)-7,11-dihydroxy-10-propyl-8,8,12,16-tetramethyl-4,17-dioxa-bicyclo[14.1.0]heptadecane-5,9-dione
(4S,7R,8S,9S,13Z,16S)-4,8-Dihydroxy-7-butyl-5,5,9,13-tetramethyl-16-(2-methyl-benzothiazol-5-yl)oxacyclohexadec-13-ene-2,6-dione
(4S,7R,8S,9S,13Z,16S)-4,8-Dihydroxy-16-(2-hydroxymethyl-benzothiazol-5-yl)-7-butyl-5,5,9,13-tetramethyl-oxacyclohexadec-13-ene-2,6-dione
(4S ,7R,8S,9S,13Z,16S)-16-(2-Aminomethyl-benzothiazol-5-yl)-4,8-dihydroxy-7-butyl-5,5,9,13-tetramethyl-oxacyclohexadec-13-ene-2,6-dione
(1S,3S,7S,10R,11S,12S,16R)-7,11-Dihydroxy-10-butyl-8,8,12,16-tetramethyl-3-(2-methyl-benzothiazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzothiazol-5-yl)-10-butyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzothiazol-5-yl)-7,11-dihydroxy-10-butyl-8,8,12,16-tetramethyl-4,17-dioxa-bicyclo[14.1.0]heptadecane-5,9-dione
(4S,7R,8S,9S,13Z,16S)-4,8-Dihydroxy-7-allyl-5,5,9,13-tetramethyl-16-(2-methyl-benzothiazol-5-yl)oxacyclohexadec-13-ene-2,6-dione
(4S,7R,8S,9S,13Z,16S)-4,8-Dihydroxy-16-(2-hydroxymethyl-benzothiazol-5-yl)-7-allyl-5,5,9,13-tetramethyl-oxacyclohexadec-13-ene-2,6-dione
(4S,7R,8S,9S,13Z,16S)-16-(2-Aminomethyl-benzothiazol-5-yl)-4,8-dihydroxy-7-allyl-5,5,9,13-tetramethyl-oxacyclohexadec-13-ene-2,6-dione
(1S,3S,7S,10R,11S,12S,16R)-7,11-Dihydroxy-10-allyl-8,8,12,16-tetramethyl-3-(2-methyl-benzothiazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzothiazol-5-yl)-10-allyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzothiazol-5-yl)-7,11-dihydroxy-10-allyl-8,8,12,16-tetramethyl-4,17-dioxa-bicyclo[14.1.0]heptadecane-5,9-dione
(4S,7R,8S,9S,13Z,16S)-4,8-Dihydroxy-7-prop-2-inyl-5,5,9,13-tetramethyl-16-(2-methyl-benzothiazol-5-yl)oxacyclohexadec-13-ene-2,6-dione
(4S,7R,8S,9S,13Z,16S)-4,8-Dihydroxy-16-(2-hydroxymethyl-benzothiazol-5-yl)-7-prop-2-inyl-5,5,9,13-tetramethyl-oxacyclohexadec-13-ene-2,6-dione
(4S,7R,8S,9S,13Z,16S)-16-(2-Aminomethyl-benzothiazol-5-yl)-4,8-dihydroxy-7-prop-2-inyl-5,5,9,13-tetramethyl-oxacyclohexadec-13-ene-2,6-dione
(1S,3S,7S,10R,11S,12S,16R)-7,11-Dihydroxy-10-prop-2-inyl-8,8,12,16-tetramethyl-3(2-methyl-benzothiazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzothiazol-5-yl)-10-prop-2-inyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzothiazol-5-yl)-7,11-dihydroxy-10-prop-2-inyl-8,8,12,16-tetramethyl-4,17-dioxa-bicyclo[14.1.0]heptadecane-5,9-dione
(4S,7R,8S,9S,13Z,16S)-4,8-Dihydroxy-7-but-3-enyl-5,5,9,13-tetramethyl-16-(2-methyl-benzothiazol-5-yl)-oxacyclohexadec-13-ene-2,6-dione
(4S,7R,8S,9S,13Z,16S)-4,8-Dihydroxy-16-(2-hydroxymethyl-benzothiazol-5-yl)-7-but-3-enyl-5,5,9,13-tetramethyl-oxacyclohexadec-13-ene-2,6-dione
(4S,7R,8S,9S,13Z,16S)-16-(2-Aminomethyl-benzothiazol-5-yl)-4,8-dihydroxy-7-but-3-enyl-5,5,9,13-tetramethyl-oxacyclohexadec-13-ene-2,6-dione
(1S,3S,7S,10R,11S,12S,16R)-7,11-Dihydroxy-10-but-3-enyl-8,8,12,16-tetramethyl-3-(2-methyl-benzothiazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzothiazol-5-yl)-10-but-3-enyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzothiazol-5-yl)-7,11-dihydroxy-10-but-3-enyl-8,8,12,16-tetramethyl-4,17-dioxa-bicyclo[14.1.0]heptadecane-5,9-dione
(4S,7R,8S,9S,13Z,16S)-4,8-Dihydroxy-7-but-3-inyl-5,5,9,13-tetramethyl-16-(2-methyl-benzothiazol-5-yl)-oxacyclohexadec-13-ene-2,6-dione
(4S,7R,8S,9S,13Z,16S)-4,8-Dihydroxy-16-(2-hydroxymethyl-benzothiazol-5-yl)-7-but-3-inyl-5,5,9,13-tetramethyl-oxacyclohexadec-13-ene-2,6-dione
(4S,7R,8S,9S,13Z,16S)-16-(2-Aminomethyl-benzothiazol-5-yl)-4,8-dihydroxy-7-but-3-inyl-5,5,9,13-tetramethyl-oxacyclohexadec-13-ene-2,6-dione
(1S,3S,7S,10R,11S,12S,16R)-7,11-Dihydroxy-10-but-3-inyl-8,8,12,16-tetramethyl-3-(2-methyl-benzothiazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzothiazol-5-yl)-10-but-3-inyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzothiazol-5-yl)-7,11-dihydroxy-10-but-3-inyl-8,8,12,16-tetramethyl-4,17-dioxa-bicyclo[14.1.0]heptadecane-5,9-dione
(4S,7R,8S,9S,13Z,16S)-4,8-Dihydroxy-5,5,7,9,13-pentamethyl-16-(2-methyl-benzoxazol-5-yl)-oxacyclohexadec-13-ene-2,6-dione
(4S,7R,8S,9S,13Z,16S)-4,8-Dihydroxy-16-(2-hydroxymethyl-benzoxazol-5-yl)-5,5,7,9,13-pentamethyl-oxacyclohexadec-13-ene-2,6-dione
(4S,7R,8S,9S,13Z,16S)-16-(2-Aminomethyl-benzoxazol-5-yl)-4,8-dihydroxy-5,5,7,9,13-pentamethyl-oxacyclohexadec-13-ene-2,6-dione
(1S,3S,7S,10R,11S,12S,16R)-7,11-Dihydroxy-8,8,10,12,16-pentamethyl-3-(2-methyl-benzoxazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzoxazol-5-yl)-8,8,10,12,16-pentamethyl-4,17-dioxa-bicyclo[14.1.0]heptadecane-5,9-dione
(1S,3S,7S,10R,11S,12S,16R)-3-(2-Aminomethyl-benzoxazol-5-yl)-7,11-dihydroxy-8,8,10,12,16-pentamethyl-4,17-dioxa-bicyclo[14.1.0]heptadecane-5,9-dione
(4S,7R,8S,9S,13Z,16S)-4,8-Dihydroxy-7-ethyl-5,5,9,13-tetramethyl-16-(2-methyl-benzoxazol-5-yl)-oxacyclohexadec-13-ene-2,6-dione
(4S,7R,8S,9S,13Z,16S)-4,8-Dihydroxy-16-(2-hydroxymethyl-benzoxazol-5-yl)-7-ethyl-5,5,9,13-tetramethyl-oxacyclohexadec-13-ene-2,6-dione
(4S,7R,8S,9S,13Z,16S)-16-(2-Aminomethyl-benzoxazol-5-yl)-4,8-dihydroxy-7-ethyl-5,5,9,13-tetramethyl-oxacyclohexadec-13-ene-2,6-dione
(1S,3S,7S,10R,11S,12S,16R)-7,11-Dihydroxy-10-ethyl-8,8,12,16-tetramethyl-3-(2-methyl-benzoxazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzoxazol-5-yl)-10-ethyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzoxazol-5-yl)-7,11-dihydroxy-10-ethyl-8,8,12,16-tetramethyl-4,17-dioxa-bicyclo[14.1.0]heptadecane-5,9-dione
(4S,7R,8S,9S,13Z,16S)-4,8-Dihydroxy-7-propyl-5,5,9,13-tetramethyl-16-(2-methyl-benzoxazol-5-yl)-oxacyclohexadec-13-ene-2,6-dione
(4S,7R,8S,9S,13Z,16S)-4,8-Dihydroxy-16-(2-hydroxymethyl-benzoxazol-5-yl)-7-propyl-5,5,9,13-tetramethyl-oxacyclohexadec-13-ene-2,6-dione
(4S,7R,8S,9S,13Z,16S)-16-(2-Aminomethyl-benzoxazol-5-yl)-4,8-dihydroxy-7-propyl-5,5,9,13-tetramethyl-oxacyclohexadec-13-ene-2,6-dione
(1S,3S,7S,10R,11S,12S,16R)-7,11-Dihydroxy-10-propyl-8,8,12,16-tetramethyl-3-(2-methyl-benzoxazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzoxazol-5-yl)-10-propyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzoxazol-5-yl)-7,11-dihydroxy-10-propyl-8,8,12,16-tetramethyl-4,17-dioxa-bicyclo[14.1.0]heptadecane-5,9-dione
(4S,7R,8S,9S,13Z,16S)-4,8-Dihydroxy-7-butyl-5,5,9,13-tetramethyl-16-(2-methyl-benzoxazol-5-yl)-oxacyclohexadec-13-ene-2,6-dione
(4S,7R,8S,9S,13Z,16S)-4,8-Dihydroxy-16-(2-hydroxymethyl-benzoxazol-5-yl)-7-butyl-5,5,9,13-tetramethyl-oxacyclohexadec-13-ene-2,6-dione
(4S,7R,8S,9S,13Z,16S)-16-(2-Aminomethyl-benzoxazol-5-yl)-4,8-dihydroxy-7-butyl-5,5,9,13-tetramethyl-oxacyclohexadec-13-ene-2,6-dione
(1S,3S,7S,10R,11S,12S,16R)-7,11-Dihydroxy-10-butyl-8,8,12,16-tetramethyl-3-(2-methyl-benzoxazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzoxazol-5-yl)-10-butyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzoxazol-5-yl)-7,11-dihydroxy-10-butyl-8,8,12,16-tetramethyl-4,17-dioxa-bicyclo[14.1.0]heptadecane-5,9-dione
(4S,7R,8S,9S,13Z,16S)-4,8-Dihydroxy-7-allyl-5,5,9,13-tetramethyl-16-(2-methyl-benzoxazol-5-yl)-oxacyclohexadec-13-ene-2,6-dione
(4S,7R,8S,9S,13Z,16S)-4,8-Dihydroxy-16-(2-hydroxymethyl-benzoxazol-5-yl)-7-allyl-5,5,9,13-tetramethyl-oxacyclohexadec-13-ene-2,6-dione
(4S,7R,8S,9S,13Z,16S)-16-(2-Aminomethyl-benzoxazol-5-yl)-4,8-dihydroxy-7-allyl-5,5,9,13-tetramethyl-oxacyclohexadec-13-ene-2,6-dione
(1S,3S,7S,10R,11S,12S,16R)-7,11-Dihydroxy-10-allyl-8,8,12,16-tetramethyl-3-(2-methyl-benzoxazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzoxazol-5-yl)-10-allyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzoxazol-5-yl)-7,11-dihydroxy-10-allyl-8,8,12,16-tetramethyl-4,17-dioxa-bicyclo[14.1.0]heptadecane-5,9-dione
(4S,7R,8S,9S,13Z,16S)-4,8-Dihydroxy-7-prop-2-inyl-5,5,9,13-tetramethyl-16-(2-methyl-benzoxazol-5-yl)-oxacyclohexadec-13-ene-2,6-dione
(4S,7R,8S,9S,13Z,16S)-4,8-Dihydroxy-16-(2-hydroxymethyl-benzoxazol-5-yl)-7-prop-2-inyl-5,5,9,13-tetramethyl-oxacyclohexadec-13-ene-2,6-dione
(4S,7R,8S,9S,13Z,16S)-16-(2-Aminomethyl-benzoxazol-5-yl)-4,8-dihydroxy-7-prop-2-inyl-5,5,9,13-tetramethyl-oxacyclohexadec-13-ene-2,6-dione
(1S,3S,7S,10R,11S,12S,16R)-7,11-Dihydroxy-10-prop-2-inyl-8,8,12,16-tetramethyl-3-(2-methyl-benzoxazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzoxazol-5-yl)-10-prop-2-inyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzoxazol-5-yl)-7,11-dihydroxy-10-prop-2-inyl-8,8,12,16-tetramethyl-4,17-dioxa-bicyclo[14.1.0]heptadecane-5,9-dione
(4S,7R,8S,9S,13Z,16S)-4,8-Dihydroxy-7-but-3-enyl-5,5,9,13-tetramethyl-16-(2-methyl-benzoxazol-5-yl)-oxacyclohexadec-13-ene-2,6-dione
(4S,7R,8S,9S,13Z,16S)-4,8-Dihydroxy-16-(2-hydroxymethyl-benzoxazol-5-yl)-7-but-3-enyl-5,5,9,13-tetramethyl-oxacyclohexadec-13-ene-2,6-dione
(4S,7R,8S,9S,13Z,16S)-16-(2-Aminomethyl-benzoxazol-5-yl)-4,8-dihydroxy-7-but-3-enyl-5,5,9,13-tetramethyl-oxacyclohexadec-13-ene-2,6-dione
(1S,3S,7S,10R,11S,12S,16R)-7,11-Dihydroxy-10-but-3-enyl-8,8,12,16-tetramethyl-3-(2-methyl-benzoxazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzoxazol-5-yl)-10-but-3-enyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzoxazol-5-yl)-7,11-dihydroxy-10-but-3-enyl-8,8,12,16-tetramethyl-4,17-dioxa-bicyclo[14.1.0]heptadecane-5,9-dione
(4S,7R,8S,9S,13Z,16S)-4,8-Dihydroxy-7-but-3-inyl-5,5,9,13-tetramethyl-16-(2-methyl-benzoxazol-5-yl)-oxacyclohexadec-13-ene-2,6-dione
(4S,7R,8S,9S,13Z,16S)-4,8-Dihydroxy-16-(2-hydroxymethyl-benzoxazol-5-yl)-7-but-3-inyl-5,5,9,13-tetramethyl-oxacyclohexadec-13-ene-2,6-dione
(4S,7R,8S,9S,13Z,16S)-16-(2-Aminomethyl-benzoxazol-5-yl)-4,8-dihydroxy-7-but-3-inyl-5,5,9,13-tetramethyl-oxacyclohexadec-13-ene-2,6-dione
(1S,3S,7S,10R,11S,12S,16R)-7,11-Dihydroxy-10-but-3-inyl-8,8,12,16-tetramethyl-3-(2-methyl-benzoxazol-5-yl)-4,17-dioxa-bicyclo[14.1.0]heptadecane-5,9-dione
(1S,3S,7S,10R,11S,12S,16R)-7,11-Dihydroxy-3-(2-hydroxymethyl-benzoxazol-5-yl)-10-but-3-inyl-8,8,12,16-tetramethyl-4,17-dioxa-bicyclo[14.1.0]heptadecane-5,9-dione
(1S,3S,7S,10R,11S,12S,16R)-3-(2-Aminomethyl-benzoxazol-5-yl)-7,11-dihydroxy-10-but-3-inyl-8,8,12,16-tetramethyl-4,17-dioxa-bicyclo[14.1.0]heptadecane-5,9-dione.

51. Pharmaceutical agents according to claim 1 for use in intravenous administration.

52. Pharmaceutical agents according to claim 51 for use in the therapy of tumors.

53. Pharmaceutical agents according to claim 51 for use in the graphic visualization of tumors.

54. Pharmaceutical agents according to claim 53 for use in the monitoring of the course of therapy in the treatment of tumors.

55. Use of pharmaceutical agents according to claim 1 for the production of a medication or diagnostic agent for intravenous administration.

56. Use of pharmaceutical agents according to claim 55 for the production of a medication for the therapy of tumors.

57. Use of pharmaceutical agents according to claim 55 for the production of a diagnostic agent for graphic visualization of tumors.

58. Use of pharmaceutical agents according to claim 57 for the production of a diagnostic agent for monitoring the course of therapy in the treatment of tumors.
